(19) United States
(12) Patent Application Publication
(10) Pub. No.: US 2025/0057762 A1
(43) Pub. Date: Feb. 20, 2025

(54) FORMULATIONS OF KINASE INHIBITORS AND PROSTANOIDS

(71) Applicant: PULMOKINE, INC., Troy, NY (US)

(72) Inventor: Lawrence S. Zisman, San Diego, CA (US)

(21) Appl. No.: 18/628,470

(22) Filed: Apr. 5, 2024

Related U.S. Application Data
(63) Continuation of application No. 16/968,530, filed on Aug. 7, 2020, now abandoned.
(60) Provisional application No. 62/628,068, filed on Feb. 8, 2018.

(51) Int. Cl. A61K 9/00; A61K 9/16; A61K 31/198; A61K 31/496; A61K 31/5575; A61K 47/24

(52) U.S. Cl. CPC A61K 9/0075; A61K 9/0043; A61K 9/0078; A61K 9/1623; A61K 31/198; A61K 31/496; A61K 31/5575; A61K 47/24

(57) ABSTRACT

The present disclosure describes a method of treating pulmonary disorders, such as pulmonary arterial hypertension, using a combination of a PDGF receptor kinase inhibitor and a prostanoid. The therapeutic formulations of the disclosure can inhibit cell growth and proliferation and target the underlying pathology of PAH.

### CROSS REFERENCE

This application claims the benefit of U.S. Provisional Application No. 62/628,068, filed Feb. 8, 2018, which is incorporated herein by reference in its entirety.

### GOVERNMENT RIGHTS

This invention was made with the support of the United States government under Contract number HL102946 by the National Institutes of Health. The government has certain rights in the invention.

### BACKGROUND

Pulmonary hypertension (PH) is a rare disorder of the pulmonary vasculature that is associated with high morbidity and mortality. The pathology of the disease includes plexiform lesions of disorganized angiogenesis and abnormal neointimal cellular proliferation, which obstruct blood flow through the pulmonary arterioles. Kinases play a critical role in cell growth and proliferation, and can be used to address the underlying pathology of PH.

### INCORPORATION BY REFERENCE

Each patent, publication, and non-patent literature cited in the application is hereby incorporated by reference in its entirety as if each was incorporated by reference individually.

### SUMMARY OF THE INVENTION

In an aspect, the present disclosure provides a pharmaceutical formulation comprising a) a prostanoid; and b) a compound of the formula:

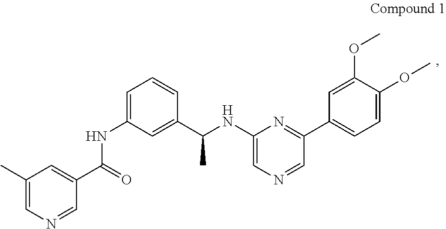

or a pharmaceutically acceptable salt thereof; wherein the pharmaceutical formulation is a spray dried powder formulation comprising a plurality of particles with a mass median aerodynamic diameter of about 1 micron to about 5 microns.

In some embodiments, the plurality of particles has a geometric standard deviation of about 1 to about 3. In some embodiments, the spray dried powder formulation has a fine particle fraction of about 70% to about 99%. In some embodiments, the pharmaceutical formulation further comprises a pharmaceutically-acceptable excipient. In some embodiments, the pharmaceutically-acceptable excipient is leucine or a pharmaceutically acceptable salt thereof. In some embodiments, the pharmaceutically-acceptable excipient is lactose. In some embodiments, the pharmaceutically-acceptable excipient is a phospholipid. In some embodiments, the prostanoid is present in an amount of about 5 μg to about 500 μg. In some embodiments, the prostanoid is present in an amount of about 6 μg to about 54 μg. In some embodiments, the prostanoid is present in an amount of about 25 μg to about 250 μg. In some embodiments, Compound 1 is present in an amount of about 46.6% w/w. In some embodiments, the prostanoid is present in an amount of about 408 μg/mg. In some embodiments, the prostanoid is Treprostinil. In some embodiments the prostanoid is epoprostenol. In some embodiments, the prostanoid is iloprost. In some embodiments, the prostanoid is beraprost. In some embodiments, the prostanoid is selexipag. In some embodiments, the prostanoid is ralinepag. In some embodiments, the prostanoid is alprostadil. In some embodiments, the prostanoid is thromboxane A2. In some embodiments, the prostanoid is thromboxane B2. In some embodiments the prostanoid is PGI₂.

In another aspect, the present disclosure provides a method of treating a pulmonary disorder comprising administering to a subject in need thereof a therapeutically effective amount of a pharmaceutical formulation comprising a) a prostanoid; and b) a compound of the formula:

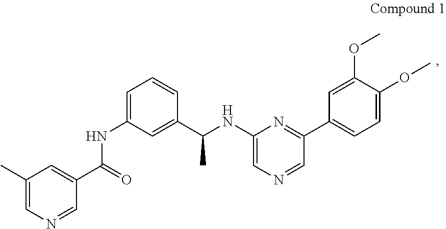

or a pharmaceutically acceptable salt thereof; wherein the pharmaceutical formulation is a spray dried powder formulation comprising a plurality of particles with a mass median aerodynamic diameter of about 1 micron to about 5 microns.

In some embodiments, the administering is by a dry powder inhaler. In some embodiments, the administering is by an atomizer. In some embodiments, the administering is by a nebulizer. In some embodiments, the administering is nasal. In some embodiments, the plurality of particles has a geometric standard deviation of about 1 to about 3. In some embodiments, the spray dried powder formulation has a fine particle fraction of about 70% to about 99%. In some embodiments, the pharmaceutical formulation further comprises a pharmaceutically-acceptable excipient. In some embodiments, the pharmaceutically-acceptable excipient is leucine or a pharmaceutically acceptable salt thereof. In some embodiments, the pharmaceutically-acceptable excipient is lactose. In some embodiments, the pharmaceutically-acceptable excipient is a phospholipid. In some embodiments, the prostanoid is present in an amount of about 5 μg to about 500 μg. In some embodiments, the prostanoid is present in an amount of about 6 μg to about 54 μg. In some embodiments, the prostanoid is present in an amount of about 25 μg to about 250 μg. In some embodiments, Compound 1 is present in an amount of about 46.6% w/w. In some embodiments, the prostanoid is present in an amount of about 408 μg/mg. In some embodiments, the prostanoid is Treprostinil. In some embodiments the prostanoid is epoprostenol. In some embodiments, the prostanoid is iloprost. In some embodiments, the prostanoid is beraprost. In some embodiments, the prostanoid is selexipag. In some embodiments, the prostanoid is ralinepag. In some embodiments, the prostanoid is alprostadil. In some embodiments the prostanoid is PGI₂.

In another aspect, the present disclosure provides a pharmaceutical formulation comprising a) leucine or a pharmaceutically acceptable salt thereof, b) Treprostinil; and c) a compound of the formula:

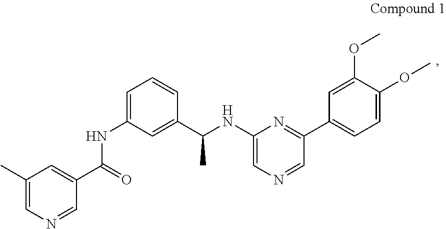

or a pharmaceutically acceptable salt thereof; wherein: i) the pharmaceutical formulation is a spray dried powder formulation comprising a plurality of particles with a mass median aerodynamic diameter of about 2.21 μm, a geometric standard deviation of about 1.79, and a fine particle fraction of about 83.6%, ii) Compound 1 is present in an amount of about 46.6% w/w; and iii) Treprostinil is present in an amount of about 408 μg/mg.

In another aspect, the present disclosure provides a method of treating a pulmonary disorder comprising nasally administering to a subject in need thereof a therapeutically effective amount of a pharmaceutical formulation comprising a) leucine or a pharmaceutically acceptable salt thereof, b) Treprostinil; and c) a compound of the formula:

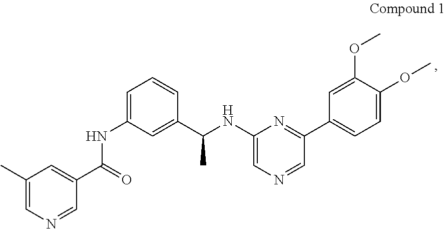

or a pharmaceutically acceptable salt thereof; wherein: i) the pharmaceutical formulation is a spray dried powder formulation comprising a plurality of particles with a mass median aerodynamic diameter of about 2.21 um, a geometric standard deviation of about 1.79, and a fine particle fraction of about 83.6%, ii) Compound 1 is present in an amount of about 46.6% w/w; and iii) Treprostinil is present in an amount of about 408 μg/mg.

### BRIEF DESCRIPTION OF THE FIGURES

FIG. 1 shows the log-probit plot that was used to calculate the mass median aerodynamic diameter and the geometric standard deviation for the spray dried powder formulation described in Example 1.

FIG. 2 shows the amount of drug per stage of the Next Generation Impactor described in Example 1.

FIG. 3 shows the cumulative drug distribution vs. the upper aerodynamic diameter for the spray dried powder formulation described in Example 1.

FIG. 4 shows a UV spectrum to determine the maximum wavelength of Treprostinil and possible interference with the blank (methanol).

FIG. 5 shows HPLC chromatograms for the blank solution (methanol) and Treprostinil.

FIG. 6 shows the calibration curve of Treprostinil. The range for the calibration curve is 0.1 to 100 μg/ml. The linear fit corresponds to an equation of y=87.9X−7.29, r=0.9999. The white circle corresponds to the points that did not meet acceptance criteria.

### DETAILED DESCRIPTION OF THE INVENTION

Pulmonary hypertension (PH), also known as pulmonary arterial hypertension (PAH), is a chronic disease that affects the arteries in the lungs and the right side of the heart. If left untreated, PAH can lead to heart failure; thus, PAH is a disorder associated with high morbidity and mortality. The World Health Organization classifies PH into five groups based on the underlying associated disease: PAH, PH due to left heart disease, PH due to lung diseases and/or hypoxia, chronic thromboembolic PH (CTEPH), and PH with other multifactorial mechanisms.

The pathology of PAH includes complex vascular formations resulting from the remodeling of pulmonary arteries called plexiform lesions and abnormal neointimal cellular proliferation, which obstruct blood flow through the pulmonary arterioles. Kinases play a critical role in cell growth and proliferation, and can be targeted to address the underlying pathology of PAH.

Signaling through the platelet derived growth factor (PDGF) pathway can promote the development and progression of PAH. The PDGF receptor (PDGFR) has two major isoforms: α and β. The α and β isoforms of PDGFR can form homodimers (i.e., PDGFRαα and PDGFRββ) and heterodimers (i.e., PDGFRαβ). In some embodiments, PDGFRαα as abbreviated as PDGFRα, and PDGFRββ is abbreviated as PDGFRβ. Signaling through the different PDGFR isoforms can activate different signaling pathways.

Ligands that bind PDGFRs are single chain proteins such as PDGFA and PDGFB, which can also form homodimers and heterodimers. Ligands that bind PDGFRα are PDGFAA, and to a lesser extent, PDGFAB and PDGFBB. PDGFBB is the primary ligand that binds PDGFRβ.

Signaling through PDGFRs plays an important role in PAH, and the PDGF pathway is activated in PAH. The PDGFRβ receptor can activate the AKT, ERK, or STAT3 pathways to promote calcium influx and angiogenesis. The PDGFRα homodimer activates the PLCγ and PI3K pathways and only stimulates calcium influx. The PDGFRαβ heterodimer activates the ERK and STAT3 pathways, stimulating NFκβ and interleukin-6 (IL-6) activity. PDGF signaling also increases transcription factors, including E2F4, Jun, ESR1, EST1, ETS1, SMAD1, SP1, STAT1, MYC, HIFA, LEF1, CEBPB, and FOS. Abbreviations: A, PDGFA; B, PDGFB; αα, PDGFRα homodimer; αβ, PDGFRαβ heterodimer; ββ, PDGFRβ homodimer; PLCγ, phospholipase C gamma; PI3K, phosphoinositide 3 kinase; ERK, extracellular related kinase (also known as p38 MAP kinase); AKT, protein kinase B; STAT3, signal transduction and activator of transcription 3.

### Compounds of the Invention

The disclosure describes therapeutic formulations of protein kinase inhibitors and methods for treating pulmonary and vascular conditions. The compounds can modulate the phosphorylation of one or more downstream targets of PDGFRα or PDGFRβ, where the downstream target is any substrate that is phosphorylated as a result of PDGFRα or PDGFRβ activation. In some embodiments, the downstream target of PDGFRα or PDGFRβ is AKT, PDGFR, STAT3, ERK1, or ERK2.

An illustrative example of a kinase inhibitor is imatinib, which is a potent PDGF inhibitor. Imatinib is less potent against the PDGFRβ isoform than the PDGFRα isoform. Imatinib decreases right ventricular systolic pressure (RVSP) by inhibiting PDGF, and improves survival in the rat monocrotaline model of PAH. Imatinib also improves cardiopulmonary hemodynamics in patients with advanced PAH. However, oral administration of imatinib is associated with significant side effects, and is not used for the treatment and prophylaxis of advanced PAH.

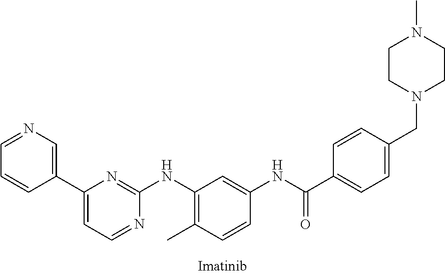 [SMILES: Cc1ccc(NC(=O)c2ccc(CN3CCN(C)CC3)cc2)cc1Nc1nccc(-c2cccnc2)n1]

In some embodiments, non-limiting illustrative examples of the kinase inhibitors disclosed herein include compounds of the following formula:

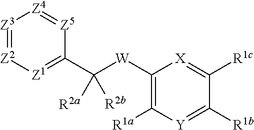

or a pharmaceutically-acceptable salt thereof,
wherein:
   W is NR¹, O, S, or a bond;
   each X and Y is independently CR² or N;
   each R^{1a}, R^{1b}, and R^{1c} is independently H, halogen, hydroxyl, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, heterocyclyl, or heteroaryl, any of which is substituted or unsubstituted;
   each R^{2a} and R^{2b} is independently H, halogen, alkyl, alkenyl, alkynyl, aryl, heterocyclyl, or heteroaryl, any of which is substituted or unsubstituted, or together form a carbonyl;
   each Z¹, Z², Z³, Z⁴, and Z⁵ is independently CR² or N; and
   each R¹ and R² is independently H, halogen, hydroxyl, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, heterocyclyl, heteroaryl, sulfhydryl, nitro, nitroso, cyano, azido, a sulfoxide group, a sulfone group, a sulfonamide group, a sulfonic acid group, an imine group, an acyl group, an acyloxy group, any of which is substituted or unsubstituted.

In some embodiments, W is NR¹, wherein R¹ is H or alkyl. In some embodiments, W is NR¹, wherein R¹ is H. In some embodiments, each R^{2a} and R^{2b} is independently H or alkyl. In some embodiments, each R^{2a} and R^{2b} is independently H or methyl. In some embodiments, each R^{2a} and R^{2b} is independently H and ethyl.

In some embodiments, each X and Y is independently CR², wherein R² is H, halogen, hydroxyl, or alkyl. In some embodiments, each X and Y is independently N. In some embodiments, X is CR², wherein R² is H, and Y is N. In some embodiments, X is N, and Y is CR², wherein R² is H.

In some embodiments, each R^{1a}, R^{1b}, and R^{1c} is independently H, halogen, hydroxyl, alkyl, aryl, or heteroaryl, any of which is substituted or unsubstituted. In some embodiments, each R^{1a}, R^{1b}, and R^{1c} is independently H, aryl, or heteroaryl, any of which is substituted or unsubstituted. In some embodiments, each R^{1a}, R^{1b}, and R^{1c} is independently H or aryl, any of which is substituted or unsubstituted. In some embodiments, each R^{1a}, R^{1b}, and R^{1c} is independently H or substituted aryl.

In some embodiments, each R^{1a} and R^{1b} is H, and R^{1c} is substituted aryl. In some embodiments, each R^{1a} and R^{1b} is H, and R^{1c} is substituted phenyl. In some embodiments, each R^{1a} and R^{1b} is H, and R^{1c} is phenyl substituted with hydroxyl, alkyl, or alkoxy. In some embodiments, each R^{1a} and R^{1b} is H, and R^{1c} is phenyl substituted with hydroxyl or alkoxy. In some embodiments, each R^{1a} and R^{1b} is H, and R^{1c} is phenyl substituted with hydroxyl and alkoxy. In some embodiments, each R^{1a} and R^{1b} is H, and R^{1c} is phenyl substituted with alkoxy. In some embodiments, each R^{1a} and R^{1b} is H, and R^{1c} is phenyl substituted with methoxy. In some embodiments, each R^{1a} and R^{1b} is H, and R^{1c} is phenyl substituted with two methoxy groups. In some embodiments, each R^{1a} and R^{1b} is H, and R^{1c} is 3,4-dimethoxyphenyl. In some embodiments, each R^{1a} and R^{1b} is H, and R^{1c} is 3-hydroxy-4-methoxyphenyl.

In some embodiments, each Z¹, Z², Z³, Z⁴, and Z⁵ is independently CR², wherein R² is H, halogen, hydroxyl, alkyl, an ether group, an amine group, or an amide group. In some embodiments, each Z¹, Z², Z³, Z⁴, and Z⁵ is independently CR², wherein R² is H or an amide group. In some embodiments, each Z¹, Z³, Z⁴, and Z⁵ is independently CR², wherein R² is H; and Z² is CR², wherein R² is an amide group. In some embodiments, each Z¹, Z³, Z⁴, and Z⁵ is independently CR², wherein R² is H; and Z² is CR², wherein R² is NHC(O)R³, wherein R³ is H, hydroxyl, alkyl, alkenyl, aryl, heterocyclyl, or heteroaryl, any of which is substituted or unsubstituted. In some embodiments, each Z¹, Z³, Z⁴, and Z⁵ is independently CR², wherein R² is H; and Z² is CR², wherein R² is NHC(O)R³, wherein R³ is aryl or heteroaryl, any of which is substituted or unsubstituted. In some embodiments, each Z¹, Z³, Z⁴, and Z⁵ is independently CR², wherein R² is H; and Z² is CR², wherein R² is NHC(O)R³, wherein R³ is substituted heteroaryl. In some embodiments, each Z¹, Z³, Z⁴, and Z⁵ is independently CR², wherein R² is H; and Z² is CR², wherein R² is NHC(O)R³, wherein R³ is substituted pyridinyl. In some embodiments, each Z¹, Z³, Z⁴, and Z⁵ is independently CR², wherein R² is H; and Z² is CR², wherein R² is NHC(O)R³, wherein R³ is methylpyridinyl. In some embodiments, Z¹, Z³, Z⁴, and Z⁵ is independently CR², wherein R² is H; and Z² is CR², wherein R² is NHC(O)R³, wherein R³ is 2-methylpyridin-5-yl.

In some embodiments, W is NR¹, wherein R¹ is H; each X and Y is independently N; each R^{1a} and R^{1b} is H; R^{1c} is substituted aryl; each R^{2a} and R^{2b} is independently H or alkyl; each Z¹, Z³, Z⁴, and Z⁵ is independently CR², wherein R² is H; Z² is CR², wherein R² is NHC(O)R³, wherein R³ is substituted heteroaryl. In some embodiments, W is NR¹, wherein R¹ is H; each X and Y is independently N; each R^{1a} and R^{1b} is H; R^{1c} is substituted phenyl; each R^{2a} and R^{2b} is independently H or alkyl; each Z¹, Z³, Z⁴, and Z⁵ is independently CR², wherein R² is H; Z² is CR², wherein R² is NHC(O)R³, wherein R³ is pyridinyl. In some embodiments, W is NR¹, wherein R¹ is H; each X and Y is independently N; each R^{1a} and R^{1b} is H; R^{1c} is phenyl with two alkoxy substituents; each R^{2a} and R^{2b} is independently H or alkyl; each Z¹, Z³, Z⁴, and Z⁵ is independently CR², wherein R² is H; Z² is CR², wherein R² is NHC(O)R³, wherein R³ is methylpyridinyl. In some embodiments, W is NR¹, wherein R¹ is H; each X and Y is independently N; each R^{1a} and R^{1b} is H; R^{1c} is phenyl substituted with an alkoxy group and a hydroxyl group; each R^{2a} and R^{2b} is independently H or alkyl; each Z¹, Z³, Z⁴, and Z⁵ is independently CR², wherein R² is H; Z² is CR², wherein R² is NHC(O)R³, wherein R³ is methylpyridinyl. In some embodiments, W is NR¹, wherein R¹ is H; each X and Y is independently N; each R^{1a} and R^{1b} is H; R^{1c} is phenyl substituted with an alkoxy group and a hydroxyl group; each R^{2a} and R^{2b} is independently H or alkyl; each Z¹, Z³, Z⁴, and Z⁵ is independently CR², wherein R² is H; Z² is CR², wherein R² is NHC(O)R³, wherein R³ is 2-methylpyridin-5-yl.

In some embodiments, non-limiting examples of the kinase inhibitors disclosed herein include compounds of the following formula:

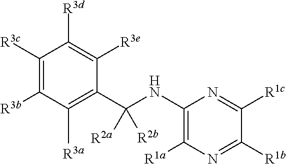

or a pharmaceutically-acceptable salt thereof,
wherein:
   each R^{1a}, R^{1b}, and R^{1c} is independently H, halogen, hydroxyl, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, heterocyclyl, or heteroaryl, any of which is substituted or unsubstituted;
   each R^{2a} and R^{2b} is independently H, halogen, alkyl, alkenyl, alkynyl, aryl, heterocyclyl, or heteroaryl, any of which is substituted or unsubstituted; and
   each R^{3a}, R^{3b}, R^{3c}, R^{3d}, and R^{3e} is independently H, halogen, hydroxyl, alkyl, alkenyl, alkynyl, an alkoxy group, an ether group, a carboxylic acid group, a carboxaldehyde group, an ester group, an amine group, an amide group, a carbonate group, a carbamate group, a thioether group, a thioester group, a thioacid group, aryl, heterocyclyl, heteroaryl, sulfhydryl, nitro, nitroso, cyano, azido, a sulfoxide group, a sulfone group, a sulfonamide group, a sulfonic acid group, an imine group, an acyl group, or an acyloxy group, any of which is substituted or unsubstituted.

In some embodiments, each R^{1a}, R^{1b}, and R^{1c} is independently H, halogen, hydroxyl, alkyl, aryl, or heteroaryl, any of which is substituted or unsubstituted. In some embodiments, each R^{1a}, R^{1b}, and R^{1c} is independently H, aryl, or heteroaryl, any of which is substituted or unsubstituted. In some embodiments, each R^{1a}, R^{1b}, and R^{1c} is independently H or aryl, wherein the aryl is substituted or unsubstituted. In some embodiments, each R^{1a} and R^{1b} is H, and R^{1c} is aryl, wherein the aryl is substituted or unsubstituted. In some embodiments, each R^{1a} and R^{1b} is H, and R^{1c} is substituted aryl. In some embodiments, each R^{1a} and R^{1b} is H, and R^{1c} is phenyl substituted with halogen, hydroxyl, alkyl, or an alkoxy group. In some embodiments, each R^{1a} and R^{1b} is H, and R^{1c} is phenyl substituted with two alkoxy groups. In some embodiments, each R^{1a} and R^{1b} is H, and R^{1c} is 3,4-dimethoxyphenyl. In some embodiments, each R^{1a} and R^{1b} is H, and R^{1c} is phenyl substituted with an alkoxy group and a hydroxyl group. In some embodiments, each R^{1a} and R^{1b} is H, and R^{1c} is 3-hydroxy-4-methoxyphenyl.

In some embodiments, each R^{2a} and R^{2b} is independently H or alkyl. In some embodiments, each R^{2a} and R^{2b} is independently H or methyl. In some embodiments, each R^{2a} and R^{2b} is independently H or ethyl.

In some embodiments, each R^{3a}, R^{3b}, R^{3c}, R^{3d}, and R^{3e} is independently H, halogen, hydroxyl, alkyl, an alkoxy group, an amine group, or an amide group, any of which is substituted or unsubstituted. In some embodiments, each R^{3a}, R^{3b}, R^{3c}, R^{3d}, and R^{3e} is independently H, hydroxyl, or an amide group, any of which is substituted or unsubstituted. In some embodiments, each R^{3a}, R^{3c}, R^{3d}, and R^{3e} is independently H, and R^{3b} is an amide group. In some embodiments, each R^{3a}, R^{3c}, R^{3d}, and R^{3e} is independently H; and R^{3b} is NHC(O)R³, wherein R³ is H, hydroxyl, alkyl, alkenyl, aryl, heterocyclyl, or heteroaryl, any of which is substituted or unsubstituted. In some embodiments, each R^{3a}, R^{3c}, R^{3d}, and R^{3e} is independently H; and R^{3b} is NHC(O)R³, wherein R³ is aryl or heteroaryl, any of which is substituted or unsubstituted. In some embodiments, each R^{3a}, R^{3c}, R^{3d}, and R^{3e} is independently H; and R^{3b} is NHC(O)R³, wherein R³ is substituted pyridinyl. In some embodiments, each R^{3a}, R^{3c}, R^{3d}, and R^{3e} is independently H; and R^{3b} is NHC(O)R³, wherein R³ is methylpyridinyl. In some embodiments, each R^{3a}, R^{3c}, R^{3d}, and R^{3e} is independently H; and R^{3b} is NHC(O)R³, wherein R³ is methylpyridin-5-yl.

In some embodiments, each R^{1a} and R^{1b} is independently H; R^{1c} is substituted aryl; each R^{2a} and R^{2b} is independently H or alkyl; each R^{3a}, R^{3c}, R^{3d}, and R^{3e} is independently H; and R^{3b} is an amide group. In some embodiments, each R^{1a} and R^{1b} is independently H; R^{1c} is substituted phenyl; each R^{2a} and R^{2b} is independently H or methyl; each R^{3a}, R^{3c}, R^{3d}, and R^{3e} is independently H; and R^{3b} is NHC(O)R³, wherein R³ is substituted heteroaryl. In some embodiments, each R^{1a} and R^{1b} is independently H; R^{1c} is substituted phenyl; each R^{2a} and R^{2b} is independently H or methyl; each R^{3a}, R^{3c}, R^{3d}, and R^{3e} is independently H; and R^{3b} is NHC(O)R³, wherein R³ is substituted pyridinyl. In some embodiments, each R^{1a} and R^{1b} is independently H; R^{1c} is substituted phenyl; each R^{2a} and R^{2b} is independently H or methyl; each R^{3a}, R^{3c}, R^{3d}, and R^{3e} is independently H; and R^{3b} is NHC(O)R³, wherein R³ is methylpyridinyl. In some embodiments, each R^{1a} and R^{1b} is independently H; R^{1c} is phenyl substituted with two alkoxy groups; each R^{2a} and R^{2b} is independently H or methyl; each R^{3a}, R^{3c}, R^{3d}, and R^{3e} is independently H; and R^{3b} is NHC(O)R³, wherein R³ is substituted pyridinyl. In some embodiments, each R^{1a} and R^{1b} is independently H; R^{1c} is phenyl substituted with one alkoxy group and one hydroxyl group; each R^{2a} and R^{2b} is independently H or methyl; each R^{3a}, R^{3c}, R^{3d}, and R^{3e} is independently H; and R^{3b} is NHC(O)R³, wherein R³ is substituted pyridinyl.

In some embodiments, non-limiting examples of the kinase inhibitors disclosed herein include compounds of the following formula:

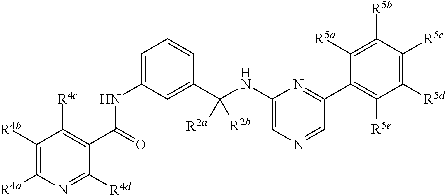

or a pharmaceutically-acceptable salt thereof,
wherein:
   each R^{2a} and R^{2b} is independently H, halogen, alkyl, alkenyl, alkynyl, aryl, heterocyclyl, or heteroaryl, any of which is substituted or unsubstituted;
   each R^{4a}, R^{4b}, R^{4c}, and R^{4d} is independently H, halogen, hydroxyl, alkyl, an alkoxy group, a carboxylic acid group, an ester group, an amine group, an amide group, sulfhydryl, nitro, nitroso, cyano, azido, a sulfoxide group, a sulfone group, a sulfonamide group, a sulfonic acid group, or an acyloxy group, any of which is substituted or unsubstituted; and
   each R^{5a}, R^{5b}, R^{5c}, R^{5d},and R^{5e} is independently H, halogen, hydroxyl, alkyl, an alkoxy group, a carboxylic acid group, ester group, an amine group, or an amide group, any of which is substituted or unsubstituted.

In some embodiments, each R^{2a} and R^{2b} is independently H or alkyl. In some embodiments, each R^{2a} and R^{2b} is independently H or methyl. In some embodiments, each R^{2a} and R^{2b} is independently H or ethyl. In some embodiments, each R^{2a} and R^{2b} independently H and ethyl.

In some embodiments, each R^{4a}, R^{4b}, R^{4c}, and R^{4d} is independently H, halogen, hydroxyl, or alkyl. In some embodiments, each R^{4a}, R^{4b}, R^{4c}, and R^{4d} is independently H or alkyl. In some embodiments, each R^{4a}, R^{4c}, and R^{4d} is independently H; and R^{4b} is alkyl. In some embodiments, each R^{4a}, R^{4c}, and R^{4d} is independently H; and R^{4b} is methyl.

In some embodiments, each R^{5a}, R^{5b}, R^{5c}, R^{5d},and R^{5e} is independently H, hydroxyl, alkoxy, or an amine group, any of which is substituted or unsubstituted. In some embodiments, each R^{5a}, R^{5b}, R^{5c}, R^{5d},and R^{5e} is independently H, hydroxyl, or alkoxy. In some embodiments, each R^{5a}, R^{5d},and R^{5e} is independently H; and each R^{5b} and R^{5c} is independently hydroxyl or alkoxy. In some embodiments, each R^{5a}, R^{5d},and R^{5e} is independently H; and each R^{5b} and R^{5c} is independently alkoxy. In some embodiments, each R^{5a}, R^{5d},and R^{5e} is independently H; and each R^{5b} and R^{5c} is independently methoxy. In some embodiments, each R^{5a}, R^{5d},and R^{5e} is independently H; R^{5b} is alkoxy; and R^{5c} hydroxyl. In some embodiments, each R^{5a}, R^{5d},and R^{5e} is independently H; R^{5b} is methoxy; and R^{5c} hydroxyl.

In some embodiments, the compounds herein are of the formula:

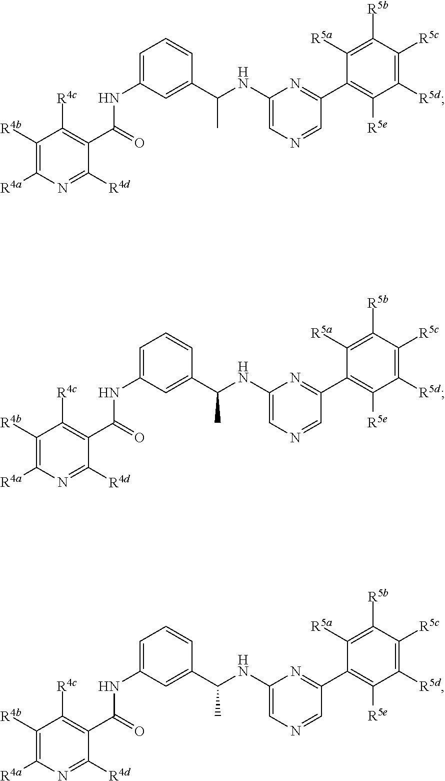

or a pharmaceutically-acceptable salt thereof, with variables defined above.

Non-limiting examples of compounds herein include the following:

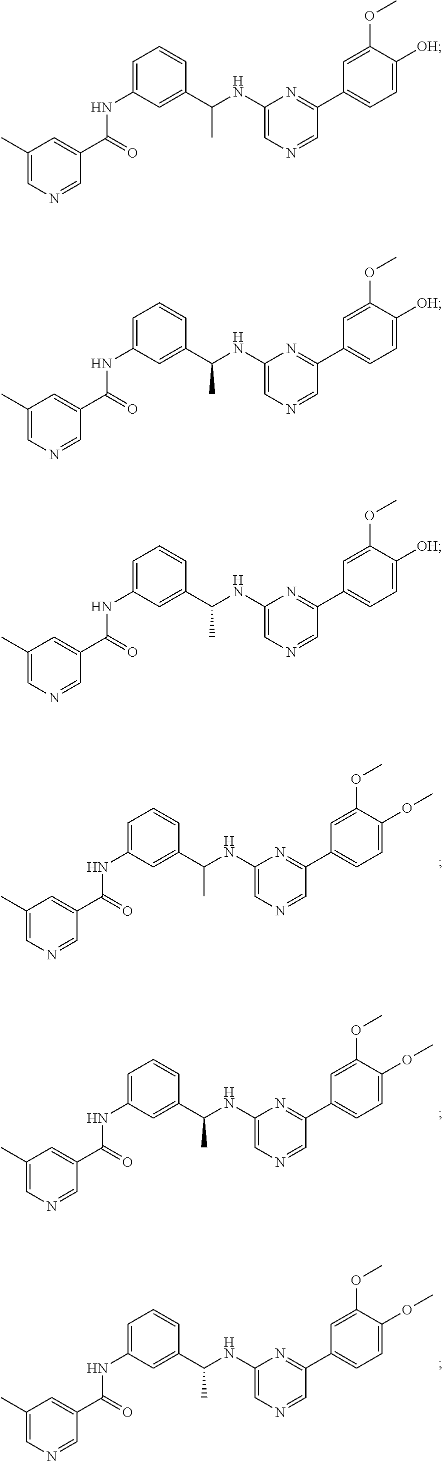

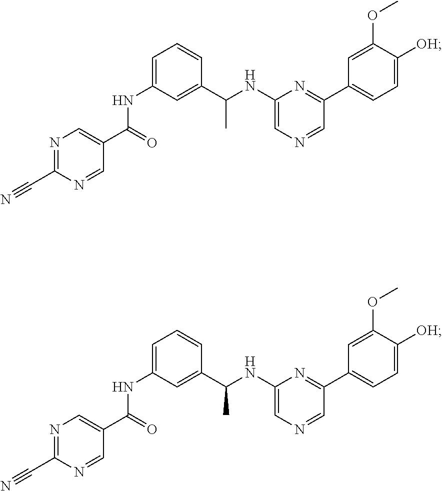 [SMILES: COc1cc(-c2cncc(NC(C)c3cccc(NC(=O)c4cnc(C#N)nc4)c3)n2)ccc1O ; COc1cc(-c2cncc(N[C@@H](C)c3cccc(NC(=O)c4cnc(C#N)nc4)c3)n2)ccc1O]

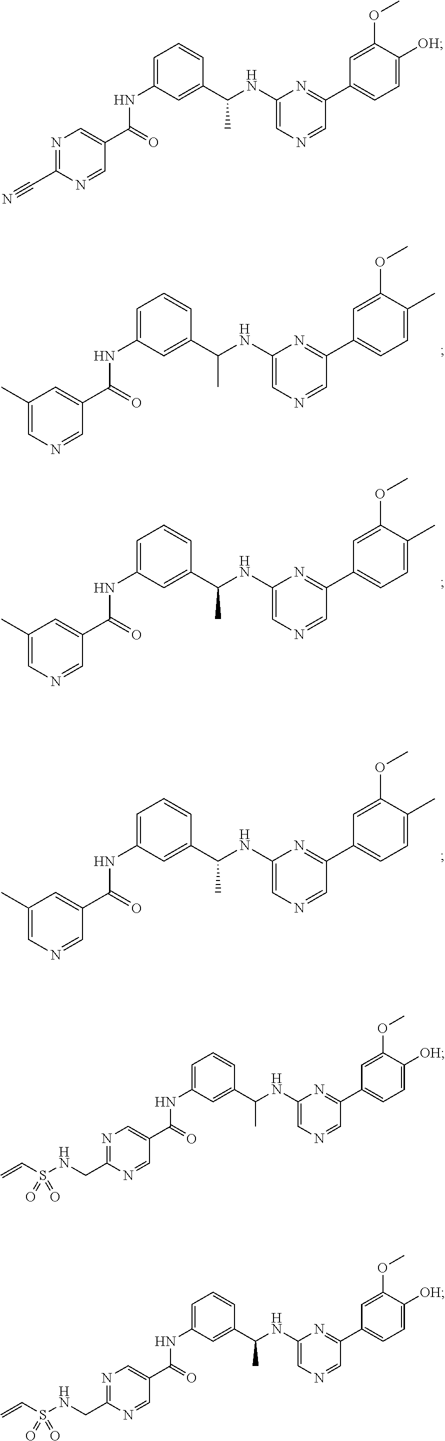

or a pharmaceutically-acceptable salt thereof.

Non-limiting examples of compounds herein include the following:

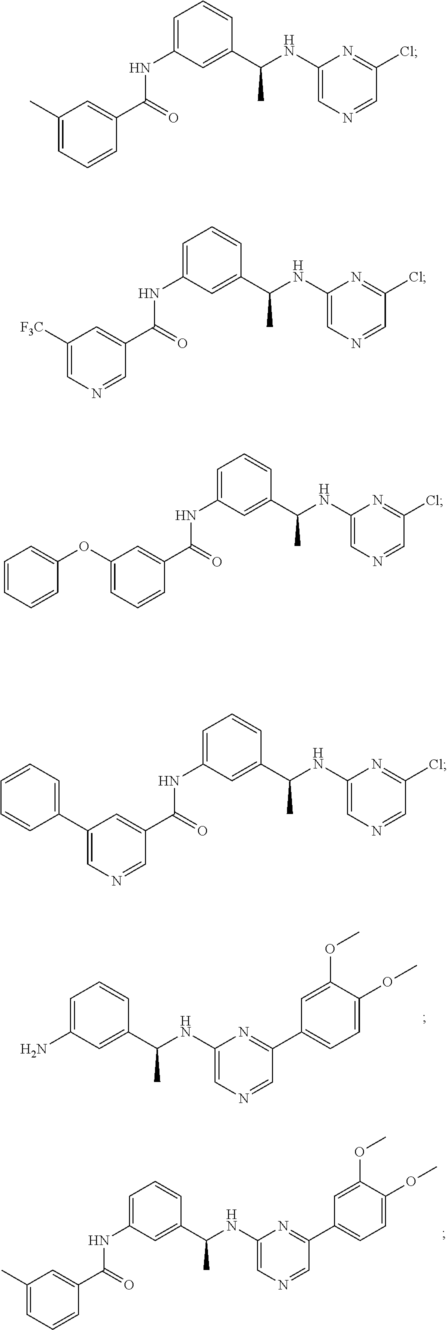

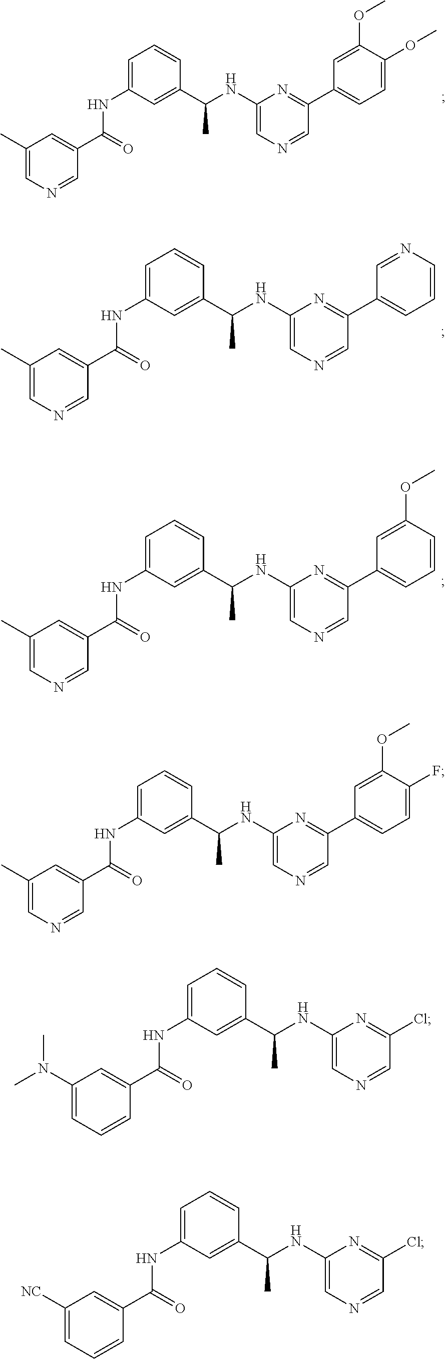

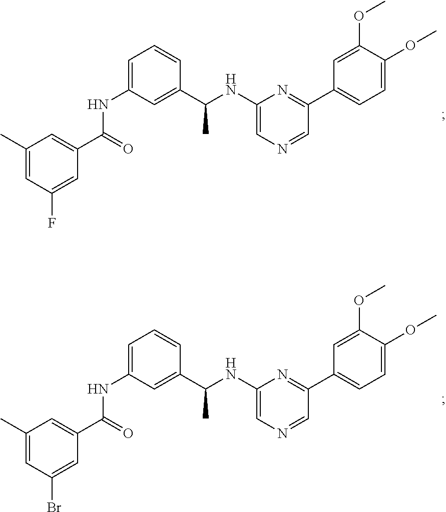

or a pharmaceutically-acceptable salt thereof.

Non-limiting examples of compounds herein include the following:

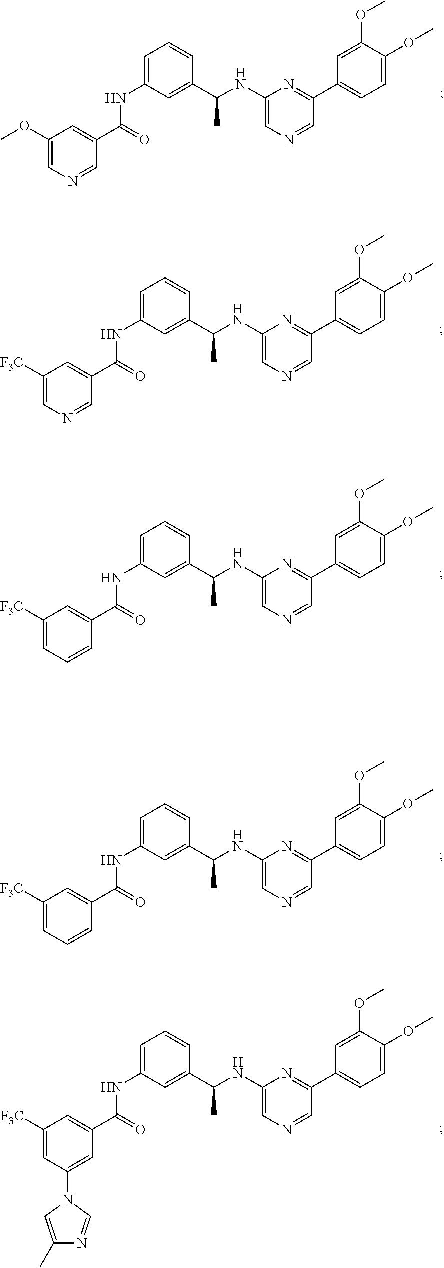

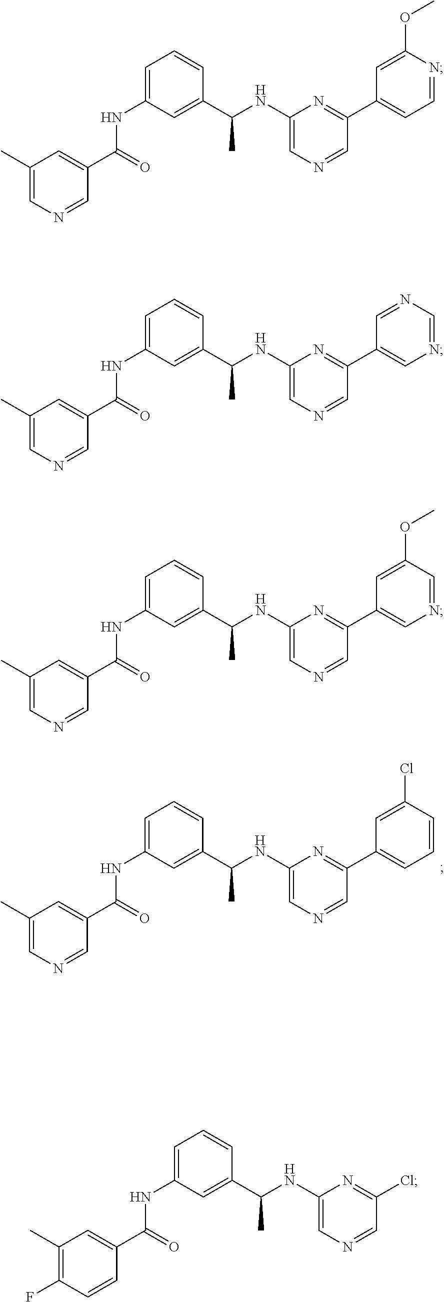

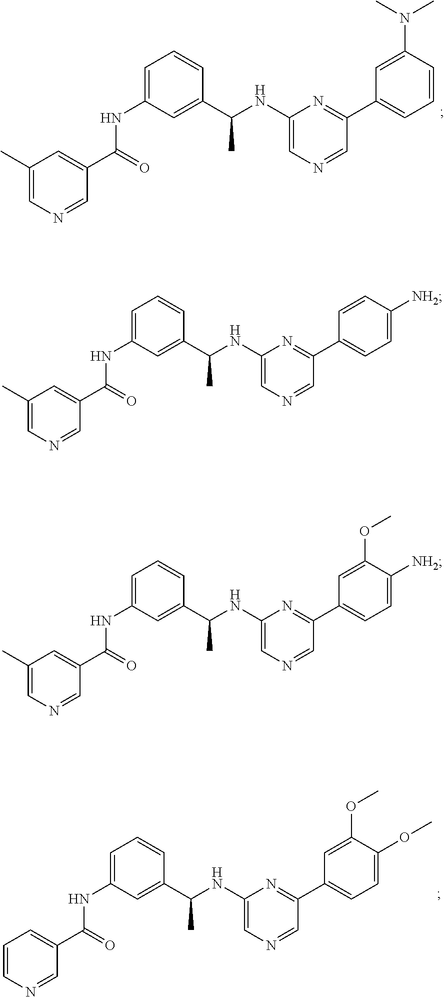

or a pharmaceutically-acceptable salt thereof.

An illustrative example of a kinase inhibitor is Compound 1, which is a compound of the formula below or a pharmaceutically-acceptable salt thereof. In some embodiments, Compound 1 and other compounds disclosed herein inhibit PDGFR (PDGFR inhibitors).

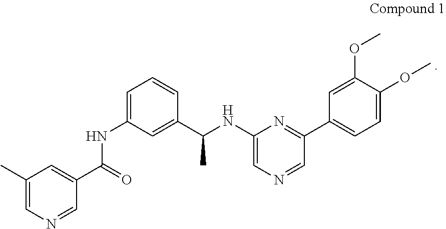

### Optional Substituents for Chemical Groups

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, and ester groups.

Non-limiting examples of alkyl and alkylene groups include straight, branched, and cyclic alkyl and alkylene groups. An alkyl or alkylene group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of cyclic alkyl groups include cyclopropyl, cyclobutyl, cyclopentyl, cyclohexyl, cycloheptlyl, and cyclooctyl groups. Cyclic alkyl groups also include fused-, bridged-, and spiro-bicycles and higher fused-, bridged-, and spiro-systems. A cyclic alkyl group can be substituted with any number of straight, branched, or cyclic alkyl groups.

Non-limiting examples of alkenyl and alkenylene groups include straight, branched, and cyclic alkenyl groups. The olefin or olefins of an alkenyl group can be, for example, E, Z, cis, trans, terminal, or exo-methylene. An alkenyl or alkenylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Non-limiting examples of alkynyl or alkynylene groups include straight, branched, and cyclic alkynyl groups. The triple bond of an alkylnyl or alkynylene group can be internal or terminal. An alkylnyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

An aryl group can be heterocyclic or non-heterocyclic. An aryl group can be monocyclic or polycyclic. An aryl group can be substituted with any number of substituents described herein, for example, hydrocarbyl groups, alkyl groups, alkoxy groups, and halogen atoms. Non-limiting examples of aryl groups include phenyl, toluyl, naphthyl, pyrrolyl, pyridyl, imidazolyl, thiophenyl, and furyl.

An aryloxy group can be, for example, an oxygen atom substituted with any aryl group, such as phenoxy.

An aralkyl group can be, for example, any alkyl group substituted with any aryl group, such as benzyl.

An arylalkoxy group can be, for example, an oxygen atom substituted with any aralkyl group, such as benzyloxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinamide, maleimide, morpholine, imidazole, thiophene, furan, tetrahydrofuran, pyran, and tetrahydropyran.

An acyl group can be, for example, a carbonyl group substituted with hydrocarbyl, alkyl, hydrocarbyloxy, alkoxy, aryl, aryloxy, aralkyl, arylalkoxy, or a heterocycle. Non-limiting examples of acyl include acetyl, benzoyl, benzyloxycarbonyl, phenoxycarbonyl, methoxycarbonyl, and ethoxy carbonyl.

An acyloxy group can be an oxygen atom substituted with an acyl group. An ester or an ester group comprises an acyloxy group. A non-limiting example of an acyloxy group, or an ester group, is acetate.

A carbamate group can be an oxygen atom substituted with a carbamoyl group, wherein the nitrogen atom of the carbamoyl group is unsubstituted, monosubstituted, or disubstituted with one or more of hydrocarbyl, alkyl, aryl, heterocyclyl, or aralkyl. When the nitrogen atom is disubstituted, the two substituents together with the nitrogen atom can form a heterocycle.

### PDGFR Inhibitors and Prostanoids

The therapeutic formulations of the disclosure can comprise a PDGFR inhibitor, such as, for example, Compound 1, and a prostanoid. The prostanoid can be any agonist of the prostaglandin I2 (IP) receptor. Prostanoids are a subclass of eicosanoids, which include prostaglandins, thromboxanes, and prostacyclins. Prostaglandins are mediators of inflammatory and anaphylactic reactions. Thromboxanes are mediators of vasoconstriction. Prostacyclins are active in the resolution phase of inflammation.

Prostaglandins are a group of physiologically active lipid compounds having diverse hormone-like effects in animals. Prostaglandins are found in almost every tissue in humans and other animals. Prostaglandins are derived enzymatically from fatty acids. Every prostaglandin contains 20 carbon atoms, including a 5-membered carbocycle. One non-limiting example of a prostaglandin that can be formulated with a PDGFR inhibitor of the disclosure is alprostadil, the structure of which is shown below.

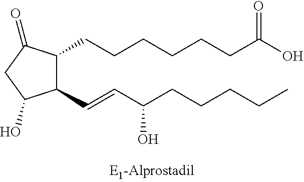 [SMILES: CCCCC[C@H](O)/C=C/[C@H]1[C@H](O)CC(=O)[C@@H]1CCCCCCC(=O)O]

In some embodiments, thromboxanes are used in a therapeutic formulation of the disclosure. Thromboxane is named for playing a role in clot formation (i.e., thrombosis). The two major thromboxanes are thromboxane A2 and thromboxane B2, the structures of which are shown below. The distinguishing feature of thromboxanes is a 6-membered ether-containing ring.

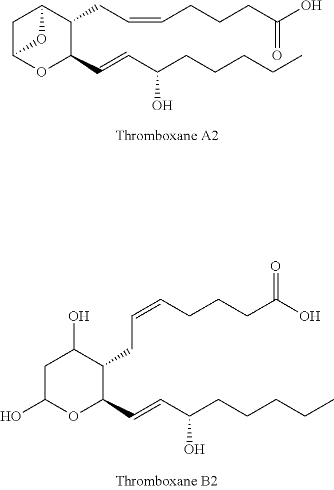 [SMILES: CCCCC[C@H](O)/C=C/[C@H]1O[C@H]2C[C@H](O2)[C@@H]1C/C=C\CCCC(=O)O ; CCCCC[C@H](O)/C=C/[C@H]1OC(O)CC(O)[C@@H]1C/C=C\CCCC(=O)O]

Prostacyclin (PGI₂) inhibits platelet activation and is also an effective vasodilator. When used as a drug, prostacyclin is also known as epoprostenol. PGI₂ prevents formation of platelet plugs involved in primary hemostasis by inhibiting platelet activation. Treprostinil is a synthetic analogue of PGI₂, and is marketed under the trade names Remodulin® for infusion and Tyvaso® for inhalation. In some embodiments, PGI₂ or Treprostinil are used in the therapeutic formulations of the disclosure. The structures of PGI₂ sodium and Treprostinil® sodium are shown below.

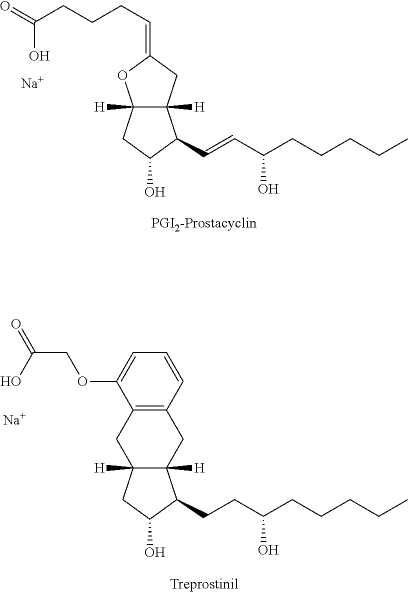 [SMILES: CCCCC[C@H](O)/C=C/[C@@H]1[C@H]2C/C(=C/CCCC(=O)O)O[C@H]2C[C@H]1O ; [Na+] ; [Na+] ; CCCCC[C@H](O)CC[C@@H]1[C@H]2Cc3cccc(OCC(=O)O)c3C[C@H]2C[C@H]1O]

In some embodiments, the prostanoids used in the therapeutic formulations of the disclosure include iloprost, beraprost, selexipag, epoprostenol, and ralinepag, the structures of each of which are shown below

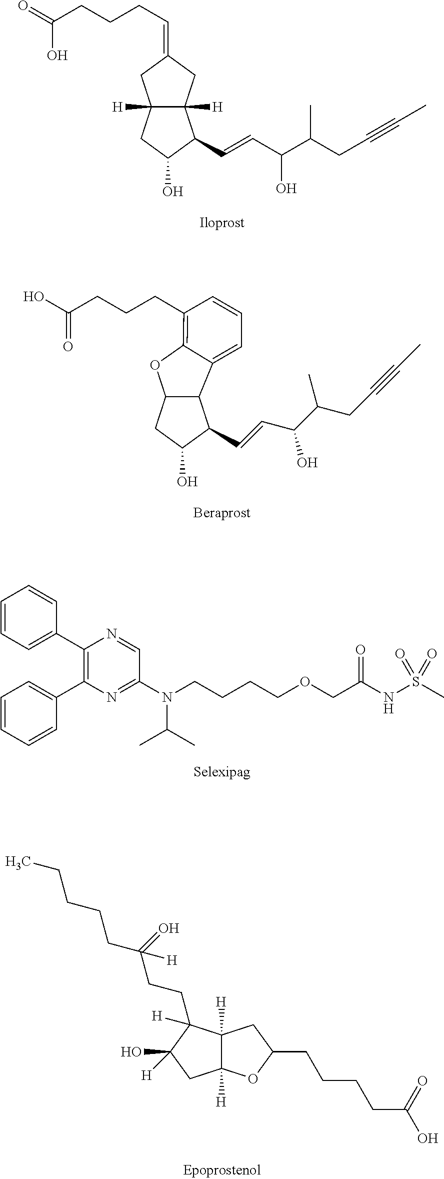

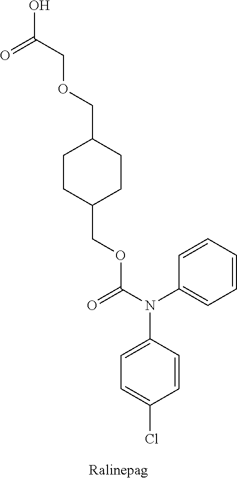 [SMILES: O=C(O)COCC1CCC(COC(=O)N(c2ccccc2)c2ccc(Cl)cc2)CC1]

### Formulations

The therapeutic formulations of the disclosure can comprise a mixture of a therapeutically-effective amount of a PDGFR inhibitor and a therapeutically-effective amount of a prostanoid. A non-limiting example of a PDGFR inhibitor is, for example, Compound 1. Non-limiting examples of prostanoids include, for example, alprostadil, thromboxane A2, thromboxane B2, PGI₂, Treprostinil, iloprost, beraprost, selexipag, epoprostenol, and ralinepag. The therapeutic formulations of the disclosure can comprise about 0.5 mg to about 500 mg of a PDGFR inhibitor. In some embodiments, the therapeutic formulations of the disclosure comprise about 0.5 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg of a PDGFR inhibitor. In some embodiments, the therapeutic formulations of the disclosure comprise about 150 mg, about 200 mg, about 250 mg, about 300 mg, about 350 mg, about 400 mg, about 450 mg, or about 500 mg of a PDGFR inhibitor.

The therapeutic formulations of the disclosure can comprise about 5 μg to about 500 μg of a prostanoid. In some embodiments, the therapeutic formulations of the disclosure comprise about 5 μg, about 10 μg, about 15 μg, about 20 μg, about 25 μg, about 30 μg, about 35 μg, about 40 μg, about 45 μg, about 50 μg, about 55 μg, about 60 μg, about 65 μg, about 70 μg, about 75 μg, about 80 μg, about 85 μg, about 90 μg, about 95 μg, or about 100 μg of a prostanoid. In some embodiments, the therapeutic formulations of the disclosure comprise about 150 μg, about 200 μg, about 250 μg, about 300 μg, about 350 μg, about 400 μg, about 450 μg, or about 500 μg of a prostanoid. In some embodiments, the therapeutic formulations of the disclosure can comprise about 6 μg to about 54 μg of a prostanoid. In some embodiments, the therapeutic formulations of the disclosure can comprise about 25 μg to about 250 μg of a prostanoid.

In some embodiments, a therapeutic formulation of the disclosure can comprise about 5% (w/w) to about 90% (w/w) of a PDGFR inhibitor disclosed herein. In some embodiments, a therapeutic formulation of the disclosure can comprise about 5% (w/w) to about 10% (w/w), about 5% (w/w) to about 20% (w/w), about 5% (w/w) to about 30% (w/w), about 5% (w/w) to about 40% (w/w), about 5% (w/w) to about 50% (w/w), about 5% (w/w) to about 60% (w/w), about 5% (w/w) to about 70% (w/w), about 5% (w/w) to about 80% (w/w), about 5% (w/w) to about 90% (w/w), about 10% (w/w) to about 20% (w/w), about 10% (w/w) to about 30% (w/w), about 10% (w/w) to about 40% (w/w), about 10% (w/w) to about 50% (w/w), about 10% (w/w) to about 60% (w/w), about 10% (w/w) to about 70% (w/w), about 10% (w/w) to about 80% (w/w), about 10% (w/w) to about 90% (w/w), about 20% (w/w) to about 30% (w/w), about 20% (w/w) to about 40% (w/w), about 20% (w/w) to about 50% (w/w), about 20% (w/w) to about 60% (w/w), about 20% (w/w) to about 70% (w/w), about 20% (w/w) to about 80% (w/w), about 20% (w/w) to about 90% (w/w), about 30% (w/w) to about 40% (w/w), about 30% (w/w) to about 50% (w/w), about 30% (w/w) to about 60% (w/w), about 30% (w/w) to about 70% (w/w), about 30% (w/w) to about 80% (w/w), about 30% (w/w) to about 90% (w/w), about 40% (w/w) to about 50% (w/w), about 40% (w/w) to about 60% (w/w), about 40% (w/w) to about 70% (w/w), about 40% (w/w) to about 80% (w/w), about 40% (w/w) to about 90% (w/w), about 50% (w/w) to about 60% (w/w), about 50% (w/w) to about 70% (w/w), about 50% (w/w) to about 80% (w/w), about 50% (w/w) to about 90% (w/w), about 60% (w/w) to about 70% (w/w), about 60% (w/w) to about 80% (w/w), about 60% (w/w) to about 90% (w/w), about 70% (w/w) to about 80% (w/w), about 70% (w/w) to about 90% (w/w), or about 80% (w/w) to about 90% (w/w) of a PDGFR inhibitor disclosed herein. In some embodiments, a therapeutic formulation of the disclosure can comprise about 5% (w/w), about 10% (w/w), about 20% (w/w), about 30% (w/w), about 40% (w/w), about 50% (w/w), about 60% (w/w), about 70% (w/w), about 80% (w/w), or about 90% (w/w) of a PDGFR inhibitor disclosed herein. In some embodiments, a therapeutic formulation of the disclosure can comprise at least about 5% (w/w), about 10% (w/w), about 20% (w/w), about 30% (w/w), about 40% (w/w), about 50% (w/w), about 60% (w/w), about 70% (w/w), or about 80% (w/w) of a PDGFR inhibitor disclosed herein. In some embodiments, a therapeutic formulation of the disclosure can comprise at most about 10% (w/w), about 20% (w/w), about 30% (w/w), about 40% (w/w), about 50% (w/w), about 60% (w/w), about 70% (w/w), about 80% (w/w), or about 90% (w/w) of a PDGRF inhibitor disclosed herein. In some embodiments, a therapeutic formulation of the disclosure can comprise about 33.7% (w/w) or about 46.6% (w/w) of a PDGFR inhibitor disclosed herein.

In some embodiments, the therapeutic formulations of the disclosure can comprise about 50 μg/mg to about 900 μg/mg of a prostanoid, such as, for example Treprostinil. In some embodiments, the therapeutic formulations of the disclosure can comprise about 50 μg/mg to about 100 μg/mg, about 50 μg/mg to about 200 μg/mg, about 50 μg/mg to about 300 μg/mg, about 50 μg/mg to about 400 μg/mg, about 50 μg/mg to about 500 μg/mg, about 50 μg/mg to about 600 μg/mg, about 50 μg/mg to about 700 μg/mg, about 50 μg/mg to about 800 μg/mg, about 50 μg/mg to about 900 μg/mg, about 100 μg/mg to about 200 μg/mg, about 100 μg/mg to about 300 μg/mg, about 100 μg/mg to about 400 μg/mg, about 100 μg/mg to about 500 μg/mg, about 100 μg/mg to about 600 μg/mg, about 100 μg/mg to about 700 μg/mg, about 100 μg/mg to about 800 μg/mg, about 100 μg/mg to about 900 μg/mg, about 200 μg/mg to about 300 μg/mg, about 200 μg/mg to about 400 μg/mg, about 200 μg/mg to about 500 μg/mg, about 200 μg/mg to about 600 μg/mg, about 200 μg/mg to about 700 μg/mg, about 200 μg/mg to about 800 μg/mg, about 200 μg/mg to about 900 μg/mg, about 300 μg/mg to about 400 μg/mg, about 300 μg/mg to about 500 μg/mg, about 300 μg/mg to about 600 μg/mg, about 300 μg/mg to about 700 μg/mg, about 300 μg/mg to about 800 μg/mg, about 300 μg/mg to about 900 μg/mg, about 400 μg/mg to about 500 μg/mg, about 400 μg/mg to about 600 μg/mg, about 400 μg/mg to about 700 μg/mg, about 400 μg/mg to about 800 μg/mg, about 400 μg/mg to about 900 μg/mg, about 500 μg/mg to about 600 μg/mg, about 500 μg/mg to about 700 μg/mg, about 500 μg/mg to about 800 μg/mg, about 500 μg/mg to about 900 μg/mg, about 600 μg/mg to about 700 μg/mg, about 600 μg/mg to about 800 μg/mg, about 600 μg/mg to about 900 μg/mg, about 700 μg/mg to about 800 μg/mg, about 700 μg/mg to about 900 μg/mg, or about 800 μg/mg to about 900 μg/mg of a prostanoid. In some embodiments, the therapeutic formulations of the disclosure can comprise about 50 μg/mg, about 100 μg/mg, about 200 μg/mg, about 300 μg/mg, about 400 μg/mg, about 500 μg/mg, about 600 μg/mg, about 700 μg/mg, about 800 μg/mg, or about 900 μg/mg of a prostanoid. In some embodiments, the therapeutic formulations of the disclosure can comprise at least about 50 μg/mg, about 100 μg/mg, about 200 μg/mg, about 300 μg/mg, about 400 μg/mg, about 500 μg/mg, about 600 μg/mg, about 700 μg/mg, or about 800 μg/mg of a prostanoid. In some embodiments, the therapeutic formulations of the disclosure can comprise at most about 100 μg/mg, about 200 μg/mg, about 300 μg/mg, about 400 μg/mg, about 500 μg/mg, about 600 μg/mg, about 700 μg/mg, about 800 μg/mg, or about 900 μg/mg of a prostanoid. In some embodiments a therapeutic formulation of the disclosure can comprise about 400 μg/mg, about 401 μg/mg, about 402 μg/mg, about 403 μg/mg, about 404 μg/mg, about 405 μg/mg, about 406 μg/mg, about 407 μg/mg, about 408 μg/mg, about 409 μg/mg, about 410 μg/mg, about 411 μg/mg, about 412 μg/mg, about 413 μg/mg, about 414 μg/mg, about 415 μg/mg, about 416 μg/mg, about 417 μg/mg, about 418 μg/mg, about 419 μg/mg, about 420 μg/mg, about 421 μg/mg, about 422 μg/mg, about 423 μg/mg, about 424 μg/mg, about 425 μg/mg, about 426 μg/mg, about 427 μg/mg, about 428 μg/mg, about 429 μg/mg, about 430 μg/mg, about 431 μg/mg, about 432 μg/mg, about 433 μg/mg, about 434 μg/mg, about 435 μg/mg, about 436 μg/mg, about 437 μg/mg, about 438 μg/mg, about 439 μg/mg, or about 440 μg/mg of a prostanoid.

In some embodiments, the therapeutic formulations of the disclosure can comprise Compound 1 and Treprostinil. In some embodiments, the therapeutic formulations of the disclosure comprise Compound 1 and epoprostenol. In some embodiments, the therapeutic formulations of the disclosure comprise Compound 1 and iloprost. In some embodiments, the therapeutic formulations of the disclosure comprise Compound 1 and beraprost. In some embodiments, the therapeutic formulations of the disclosure comprise Compound 1 and selexipag. In some embodiments, the therapeutic formulations of the disclosure comprise Compound 1 and ralinepag. In some embodiments the therapeutic formulations of the disclosure comprise Compound 1 and alprostadil. In some embodiments the therapeutic formulations of the disclosure comprise Compound 1 and thromboxane A2. In some embodiments the therapeutic formulations of the disclosure comprise Compound 1 and thromboxane B2. In some embodiments the therapeutic formulations of the disclosure comprise Compound 1 and PGI₂.

In some embodiments, the therapeutic formulations of the disclosure can comprise imatinib and treprostinil. In some embodiments, the therapeutic formulations of the disclosure comprise imatinib and epoprostenol. In some embodiments, the therapeutic formulations of the disclosure comprise imatinib and iloprost. In some embodiments, the therapeutic formulations of the disclosure comprise imatinib and beraprost. In some embodiments, the therapeutic formulations of the disclosure comprise imatinib and selexipag. In some embodiments, the therapeutic formulations of the disclosure comprise imatinib and ralinepag. In some embodiments the therapeutic formulations of the disclosure comprise imatinib and alprostadil. In some embodiments the therapeutic formulations of the disclosure comprise imatinib and thromboxane A2. In some embodiments the therapeutic formulations of the disclosure comprise imatinib and thromboxane B2. In some embodiments the therapeutic formulations of the disclosure comprise imatinib and PGI₂.

### Pharmaceutically-Acceptable Salts

The disclosure provides the use of pharmaceutically-acceptable salts of any therapeutic compound described herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Salts derived from appropriate bases include alkali metal (e.g., sodium), alkaline earth metal, ammonium and N-(alkyl)₄⁺ salts. Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc. In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine. In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, a pipyrrazole salt, an imidazole salt, a pyrazine salt, or a pipyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid. In some embodiments, the acid salt is an acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate or an undecanoate.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

Base addition salts can arise from the addition of a base to a compound of the invention. In some embodiments, the base is sodium hydroxide, potassium hydroxide, lye, calcium hydroxide, or magnesium hydroxide. In some embodiments, the base is an alkali metasilicate, alkali metal hydroxide, sodium carbonate, sodium bicarbonate, sodium percarbonate, sodium persilicate, or potassium metabisulfite.

### Purity of Compounds of the Invention

Any compound herein can be purified. A compound herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

### Pharmaceutical Formulations of the Invention

A pharmaceutical formulation of the invention can be a combination of any pharmaceutical compounds described herein with other chemical components, such as carriers, stabilizers, diluents, dispersing agents, suspending agents, thickening agents, and/or excipients. The pharmaceutical formulation facilitates administration of the compound to an organism. Pharmaceutical formulations can be administered in therapeutically-effective amounts as pharmaceutical formulations by various forms and routes including, for example, intravenous, subcutaneous, intramuscular, oral, parenteral, ophthalmic, subcutaneous, transdermal, nasal, vaginal, and topical administration.

A pharmaceutical formulation can be administered in a local manner, for example, via injection of the compound directly into an organ, optionally in a depot or sustained release formulation or implant. Pharmaceutical formulations can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical formulations can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino) propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

The formulations can be formulated for inhalation of the formulation. In some embodiments, the compounds are administered through intranasal administration. In some embodiments, the compounds are administered as a solution, suspension, or a dry powder.

A pharmaceutical formulation of the disclosure can be administered directly to the respiratory track as an aerosol. In some embodiments, the formulations are packaged in a pressurized aerosol container with suitable propellants and adjuvants. In some embodiments, the propellants are hydrocarbon propellants, such as propane, butane, or isobutene. In some embodiments, aerosol formulations can include other ingredients, such as co-solvents, stabilizers, surfactants, antioxidants, lubricants, and pH adjusters. The aerosol formulations can be administered using a metered dose inhaler.

A pharmaceutical formulation of the disclosure can be administered in the form of a lung surfactant formulation. In some embodiments, the lung surfactant formulation is Infrasurf®, Survanta®, Curosurf®, or synthetic pulmonary surfactant formulations, such as Exosurf® and artificial lung expanding compounds (ALECs). In some embodiments, the surfactant formulations are administered via airway instillation (i.e., after intubation) or intratracheally.

A pharmaceutical formulation of the disclosure can be administered as an inhalable powder. In some embodiments, the formulations can be administered as an inhalable dry powder. In some embodiments, the powder formulation can include pharmaceutically acceptable excipients, such as monosaccharides (e.g., glucose, arabinose), disaccharides (e.g., lactose, saccharose, maltose), oligosaccharides or polysaccharides (e.g., dextrane), polyalcohols (e.g., sorbitol, mannitol, xylitol), salts (e.g., sodium chloride, calcium carbonate), or any combination thereof. In some embodiments, the formulations are administered in a non-pressurized form using a nebulizer or an atomizer.

In some embodiments, a formulation disclosed herein is administered by inhalation. Delivery of formulations disclosed herein as an inhaled dry powder results in delivery locally to the lung, resulting in lower systemic drug exposure and fewer side effects. In some embodiments, lower systemic drug exposure can lower the risk of bleeding, gastrointestinal side effects, liver toxicity, fluid retention or edema, neutropenia or leukopenia, anemia, or infection. In some embodiments, lower systemic drug exposure can lower the risk of gastrointestinal side effects, such as nausea, vomiting, or diarrhea.

In some embodiments, an inhaled dry powder formulation disclosed herein can contain about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of Compound 1 by weight. In some embodiments, the inhaled dry powder formulation can contain about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a prostanoid, such as, for example Treprostinil, by weight. In some embodiments, the inhaled dry powder formulation can comprise about 46.6% of Compound 1 by weight, and about 42% of a prostanoid, such as, for example, Treprostinil by weight.

In some embodiments, an inhaled dry powder formulation disclosed herein can contain about 1%, about 5%, about 10%, about 20%, about 30%, about 40%, about 50%, about 60%, or about 70% of a hydrophobic amino acid, such as, for example, leucine.

In some embodiments, an inhaled dry powder formulation disclosed herein can contain about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a lipid-based surfactant. In some embodiments, the inhaled dry powder formulation can contain 1,2-distearoyl-sn-glycero-3-phosphocholine (DSPC), dipalmitoylphosphatidylcholine (DPPC), 1,2-dimyristoyl-sn-glycero-3-phosphorylcholine (DMPC), or liposomes.

In practicing the methods of treatment or use provided herein, therapeutically-effective amounts of the compounds described herein are administered in pharmaceutical formulations to a subject having a disease or condition to be treated. In some embodiments, the subject is a mammal such as a human. A therapeutically-effective amount can vary widely depending on the severity of the disease, the age and relative health of the subject, the potency of the compounds used, and other factors.

Pharmaceutical formulations can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of formulations described herein include formulating compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

In some embodiments, a formulation disclosed herein can comprise a hydrophobic amino acid selected from the group consisting of tryptophan, tyrosine, leucine, trileucine, isoleucine, and phenylalanine. In some embodiments, the formulation comprises about 1%, about 5%, about 10%, about 20%, about 30%, about 40%, about 50%, about 60%, or about 70% of a hydrophobic amino acid by weight of the composition. In some embodiments, the formulations of the invention comprise leucine. In some embodiments, the formulation comprises about 5%, about 10%, about 20%, about 30%, about 40%, about 50%, about 60%, or about 70% of leucine by weight of the composition. In some embodiments, the formulation comprises about 13% of leucine by weight of the composition. In some embodiments, the formulations of the invention comprise trileucine. In some embodiments, the formulation comprises about 5%, about 10%, about 20%, about 30%, about 40%, about 50%, about 60%, or about 70% of trileucine by weight of the composition. In some embodiments, the formulation comprises about 13% of trileucine by weight of the composition.

In some embodiments, a formulation comprises a lipid product, for example, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95% of a lipid by weight of the composition. In some embodiments, the formulation comprises DSPC, DPPC, DMPC, or liposomes. In some embodiments, the formulation comprises about 30%, about 35%, about 40%, about 45%, about 50%, about 55%, about 60%, about 65%, or about 70% of DSPC by weight of the composition. In some embodiments, the formulation comprises about 30%, about 35%, about 40%, about 45%, about 50%, about 55%, about 60%, about 65%, or about 70% of DPPC by weight of the composition. In some embodiments, the formulation comprises about 30%, about 35%, about 40%, about 45%, about 50%, about 55%, about 60%, about 65%, or about 70% of DMPC by weight of the composition.

In some embodiments, the therapeutic formulations of the disclosure can be formulated as a powder containing a PDGFR inhibitor, a prostanoid, and an excipient. In some embodiments, the therapeutic formulations of the disclosure comprise leucine. In some embodiments, the therapeutic formulation comprises lactose or phospholipids as an excipient. In some embodiments, the therapeutic formulations combine a PDGFR inhibitor and a prostanoid using liposomes.

In some embodiments, the therapeutic formulations of the disclosure are manufactured by spray drying. A spray dried powder formulation of the disclosure can comprise, for example, Compound 1, Treprostinil, and leucine, and can have particle sizes suitable for inhalation. In some embodiments, the mass median aerodynamic diameter (MMAD) of the particles as measured by cascade impaction with a Next Generation Impactor (NGI) can be in the range of 1.9-3.8 microns (um) with a geometric standard deviation (GSD) 1.5-3.5. In some embodiments, the MMAD of the particles as measured by cascade impaction with a NGI can be from about 1 micron to about 5 microns with a GSD from about 1 to about 3. In some embodiments, MMAD is about 1 micron, about 1.5 microns, about 2 microns, about 2.5 microns, about 3 microns, about 3.5 microns, about 4 microns, about 4.5 microns, about 5 microns, about 5.5 microns, about 6 microns, about 6.5 microns, about 7 microns, about 7.5 microns, about 8 microns, about 8.5 microns, about 9 microns, about 9.5 microns, about 10 microns, about 11 microns, about 12 microns, about 13 microns, about 14 microns, about 15 microns, about 16 microns, about 17 microns, about 18 microns, about 19 microns, or about 20 microns. In some embodiments, the MMAD is about 1 micron. In some embodiments, the MMAD is about 2 microns to about 2.5 microns. In some embodiments, the MMAD is about 2 microns. In some embodiments, the MMAD is about 2.5 microns. In some embodiments the MMAD is about 1.59 microns. In some embodiments the MMAD is about 2.21 microns. In some embodiments, the MMAD is less than about 5 microns.

In some embodiments the GSD is about 1.5-3.5. In some embodiments the GSD is about 1.25, about 1.5, about 1.75, about 2, about 2.25, about 2.5, about 2.75, or about 3. In some embodiments the GSD is about 1.79. In some embodiments the GSD is about 1.87. In some embodiments the GSD is less than about 3.

The fine particle fraction of a powder formulation is the amount of active particles with an MMAD less than 5 μm. In some embodiments the fine particle fraction of a spray dried powder formulation of the disclosure can be, for example, about 70% to about 99%. In some embodiments the fine particle fraction of a spray dried powder formulation of the disclosure can be about 70% to about 75%, about 70% to about 80%, about 70% to about 85%, about 70% to about 90%, about 70% to about 95%, about 70% to about 100%, about 75% to about 80%, about 75% to about 85%, about 75% to about 90%, about 75% to about 95%, about 75% to about 100%, about 80% to about 85%, about 80% to about 90%, about 80% to about 95%, about 80% to about 99%, about 85% to about 90%, about 85% to about 95%, about 85% to about 99%, about 90% to about 95%, about 90% to about 99%, or about 95% to about 99%. In some embodiments the fine particle fraction of a spray dried powder formulation of the disclosure can be about 70%, about 75%, about 80%, about 85%, about 90%, about 95%, or about 99%. In some embodiments the fine particle fraction of a spray dried powder formulation of the disclosure can be at least about 70%, about 75%, about 80%, about 85%, about 90%, or about 95%. In some embodiments the fine particle fraction of a spray dried powder formulation of the disclosure can be at most about 75%, about 80%, about 85%, about 90%, about 95%, or about 99%. In some embodiments the fine particle fraction of a spray dried powder formulation of the disclosure can be about 79.4% or about 83.6%.

In some embodiments, a dry powder therapeutic formulation can be delivered using a dry powder inhaler, an atomizer, or a nebulizer.

### Methods of Administration

Multiple therapeutic agents can be administered in any order or simultaneously. In some embodiments, Compound 1 is administered in combination with, before, or after additional therapeutic agents. In some embodiments, the additional therapeutic agent is a prostanoid. In some embodiments the additional therapeutic agent is Treprostinil. If administered simultaneously, the multiple therapeutic agents can be provided in a single, unified form, or in multiple forms, for example, as multiple separate pills, or in a single spray dried formulation. The agents can be packed together or separately, in a single package or in a plurality of packages. One or all of the therapeutic agents can be given in multiple doses. If not simultaneous, the timing between the multiple doses can vary to as much as about a month.

In some embodiments, Compound 1 is administered before a second agent. In some embodiments, Compound 1 is administered before administration of a prostanoid. In some embodiments, Compound 1 is administered before administration of Treprostinil. In some embodiments, Compound 1 is administered after administration of a first agent. In some embodiments, Compound 1 is administered after administration of a prostanoid. In some embodiments, Compound 1 is administered after administration of Treprostinil.

Pharmaceutical formulations described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical formulations provided herein can be administered in conjunction with other therapies, for example, chemotherapy, radiation, surgery, anti-inflammatory agents, and selected vitamins. The other agents can be administered prior to, after, or concomitantly with the pharmaceutical compositions.

Compositions of the invention can be packaged as a kit. In some embodiments, a kit includes written instructions on the administration/use of the composition. The written material can be, for example, a label. The written material can suggest methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies. In some embodiments, a kit includes an inhalation delivery device, such as an inhaler, an atomizer, or a nebulizer. In some embodiments, a kit includes inhalation capsules of powders in a sealed blister pack.

### Dosing

Pharmaceutical formulations described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein, such as, for example, Compound 1, can be present in a formulation in a range of from about 1 mg to about 500 mg. A compound described herein, such as, for example, Compound 1 can be present in a formulation in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, or about 500 mg. In some embodiments a formulation of the disclosure also comprises a prostanoid and leucine. In some embodiments a formulation of the disclosure also comprises Treprostinil and leucine.

In some embodiments a prostanoid can be present in a formulation in a range of from about 1 mg to about 500 mg. A prostanoid can be present in a formulation in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, or about 500 mg. In some embodiments a formulation of the disclosure also comprises Compound 1 and leucine.

In some embodiments Treprostinil can be present in a formulation in a range of from about 1 mg to about 500 mg. Treprostinil can be present in a formulation in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, or about 500 mg. In some embodiments a composition of the disclosure also comprises Compound 1 and leucine.

In some embodiments, a dose of Compound 1 can be about 2.5 mg to about 100 mg, In some embodiments, a dose of Compound 1 can be about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg. In some embodiments, a dose of Compound 1 can be about 0. 1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, or about 0.9 mg/kg. In some embodiments, the invention describes administration of about 0.5 mg/kg to about 0.6 mg/kg of Compound 1. In some embodiments, a dose of Compound 1 can be about 10 mg. In some embodiments, a dose of Compound 1 can be administered once, twice, three times, or four times a day.

In some embodiments, a dose of a prostanoid, such as, for example, Treprostinil can be about 5 mg, about 10 mg, about 15 mg, about 20 mg, about 25 mg, about 30 mg, about 35 mg, about 40 mg, about 45 mg, or about 50 mg. In some embodiments a dose of a prostanoid can be about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg. In some embodiments, a dose of a prostanoid can be about 5 mg. In some embodiments, a dose of a prostanoid can be about 10 mg. In some embodiments, a dose of a prostanoid can be administered once, twice, three times, or four times a day.

In some embodiments, the invention describes administration of about 1 mg/kg, about 5 mg/kg, about 10 mg/kg, about 15 mg/kg, or about 20 mg/kg of a formulation comprising Compound 1 and a prostanoid. In some embodiments, the invention describes administration of about 1 mg/kg, about 5 mg/kg, about 10 mg/kg, about 15 mg/kg, or about 20 mg/kg of a formulation comprising Compound 1 and Treprostinil.

### Indications

The therapeutic formulations of the disclosure can be used to treat diseases of the lung. In some embodiments, the condition is a pulmonary disorder, for example, PAH, PH due to left heart disease, PH due to lung disease, PH due to blood clots in the lungs, or PH resulting from blood and other rare disorders.

In some embodiments, the disclosure describes the use of a combination of compounds to treat PAH. In some embodiments, the PAH is primary PAH, idiopathic PAH, heritable PAH, drug and toxin-induced PAH, or PAH associated with other systemic diseases. In some embodiments, heritable PAH is caused by BMPR2, ALK1, endoglin, SMAD9, CAV1, or KCNK3. In some embodiments, the drug and toxin-induced PAH is induced by use of amphetamines, methamphetamines, cocaine, or fenfluramine-phentermine. In some embodiments, PAH is associated with other systemic diseases and is caused by a connective tissue disease (e.g., scleroderma, systemic lupus erythematosus, mixed connective tissue disease, and rheumatoid arthritis), human immunodeficiency virus (HIV) infection, portal hypertension, or congenital heart disease. In some embodiments, the disclosure can be used to treat pulmonary veno-occlusive disease (PVOD) or pulmonary capillary hemangiomatosis (PCH).

In some embodiments, the PH is due to left heart disease, for example, left heart disease caused by left ventricular systolic dysfunction, left ventricular diastolic dysfunction, valvular heart disease, left heart inflow and outflow obstructions not due to valvular disease, or congenital cardiomyopathies. In some embodiments, the PH is due to lung disease, for example, chronic obstructive pulmonary disease (COPD), interstitial lung diseases, sleep-disordered breathing (e.g., sleep apnea), alveolar hypoventilation disorders, chronic high altitude exposure, or developmental abnormalities of the lung.

In some embodiments, the PH is CTEPH. In some embodiments, the PH is PH with unclear or multifactorial mechanisms, such as PH caused by hematologic disorders (e.g., certain types of anemia, myeloproliferative disorders, or splenectomy), systemic disorders that have lung involvement (e.g., sarcoidosis, Langerhan cell histiocytosis, neurofibromatosis, vasculitis, or lymphangioleiomyomatosis), metabolic disorders (e.g., rare diseases of impaired cell metabolism or thyroid disease), or other unclassified diseases (e.g., chronic renal failure, tumors obstructing the pulmonary arteries, and other rare diseases).

In some embodiments, the condition to be treated is a pulmonary disorder associated with abnormal right ventricular systolic pressure (RVSP), pulmonary pressure, cardiac output, right ventricular hypertrophy, or pulmonary arterial hypertrophy. In some embodiments, the condition to be treated is lung cancer. In some embodiments, the condition to be treated is pulmonary angiosarcoma.

In some embodiments, a formulation disclosed herein reduces pulmonary pressure associated with an increase in one or more of right ventricular (RV) function, pulmonary artery (PA) systolic pressure, and/or cardiac output in the subject compared to the subject prior to the administering. In some embodiments, the reduction in pulmonary pressure is associated with a decrease in one or more of RV hypertrophy, PA hypertrophy, RVSP, sustained PA pressure, and the risk of stroke in the subject compared to the subject prior to the administering. In some embodiments, the decrease is at least a 5, 10, 15, 20, 25, 30, 35, 40, 45, 50, 55, 60, 65, 70, 75, 80, 85, 90, or 95% decrease. In some embodiments, the decrease is at least a 40% decrease.

A reduction in pulmonary pressure, in some embodiments, is not associated with decreased lung function and/or increased systemic blood pressure in the subject compared to the subject prior to the administering of the formulation. In one aspect, the present disclosure provides a composition or method of treating pulmonary arterial hypertension (PAH) in a subject, comprising: modulating the phosphory lation-state (“PS”) of one or more downstream targets of platelet derived growth factor receptor-alpha or platelet derived growth factor receptor-beta or both, wherein the downstream target is any substrate phosphorylated as a result of the PDGFR-α and/or the PDGFR-β activation, by administering to the subject a formulation comprising Compound 1 or a pharmaceutically acceptable salt thereof, Treprostinil, and leucine, wherein the downstream target is selected from the group consisting of AKT, PDGFR, STAT3, ERK1 and ERK2, or any other downstream target of the PDGFR-α and/or the PDGFR-β, and wherein the formulation is described herein.

### EXAMPLES

### Example 1—Manufacturing of a Spray Dried Pharmaceutical Formulation

Spray drying manufacturing process: A Compound 1/Treprostinil solution was prepared by adding 618.3 mg of Compound 1 in 50 ml of reagent ethanol (VWR-BDH1156-4LP. Lot #17L016505) in a clean Pyrex 125 ml Erlenmeyer flask to get a concentration of 12.37 mg/ml. The solution was agitated until clear. Throughout the preparation the solution was held at ambient temperature (72.8° F., 16% humidity). Following preparation the solution was spray-dried on a Yamoto Spray dryer (Model: ADL311 (S), Serial Number #J1307004) in open loop mode through a one-fluid nozzle (Spraying Systems Co, 1/8J 1650 liquid cap, 120 air cap).

During the spray drier warm-up, reagent ethanol and water were run through spray dryer tubes and nozzles. The two solutions were simultaneously pumped via two separate peristaltic pumps (Masterflex C/L Model: 77120-52 Serial #M17002118 and Serial number #H17002570) with a flow rate of 1 ml/min each (on max setting, through silicone feed tubes: Yamato LT00027796, OD: 3.9 mm; ID: 2.0 mm) into a single feeding tube where two solutions were mixed before entering the nozzle. A medical grade nitrogen (supplier: Noble Gas Solutions, Albany, NY) was used as atomizing gas to prevent possible burning of high concentrated ethanol in the solution mix. The setting of the spray dryer was as follows:
Atomizing gas pressure: 0.3 mPA=43.5 psi
Solution feed rate: 2.0 ml/minute
Inlet temperature: 110° C.
Outlet temperature: 60° C.

The Compound 1/Treprostinil solution in reagent ethanol (12.37 mg/ml Compound 1 and 122.1 μg/ml Treprostinil) and L-Leucine (12.26 mg/ml in water) were simultaneously pumped via two peristaltic pumps into a single feeding tube where two solutions were mixed before entering the nozzle. A high efficiency cyclone with a 1.9 inch outer diameter was used for powder collection. The spray dryer outlet ductwork was put into a chemical fume hood.

Packaging: Spray dried powder was collected off of the cyclone into an attached 600 cc glass jar. After finishing the spray drying process, the machine is cooled down to an outlet temperature of 35° C. After the cooling, the powder from the glass jar was scraped off using a cell scraper (Celltreat, Product Code: 229310) and transferred into an 8 cc amber glass bottle with a screw cap at ambient room temperature (72.8° F., 16.0% humidity). A total of 641.6 mg of spray dried powder was collected to bring up a yield of 51.8%. The spray dried powder was stored in a bottle cap sealed with parafilm, which was placed in a 50 ml centrifuge tube (Drierite, Stock #21001) with desiccant.

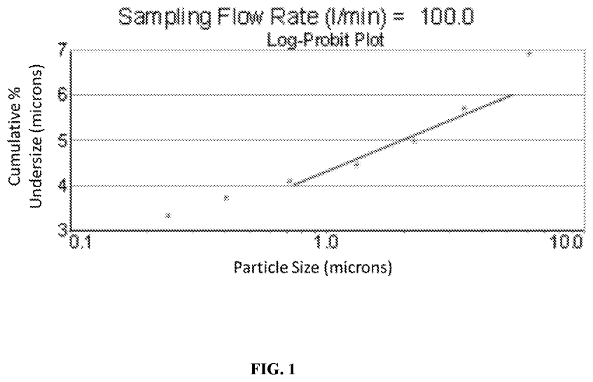

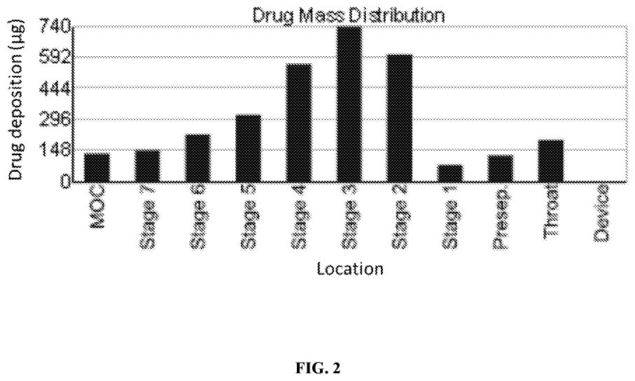

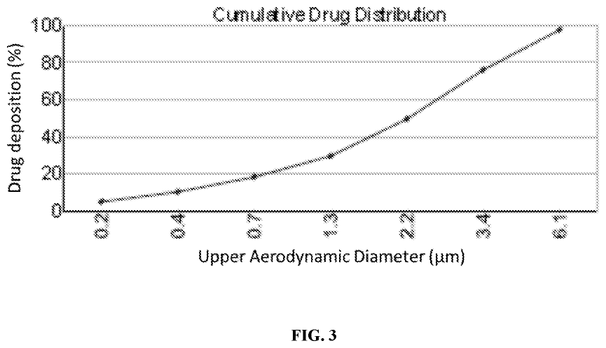

Particle characterization: The aerosol properties of the spray dried powder formulation were measured via cascade impaction with a Next Generation Impactor at a flow rate of 100 L/min. The spray dried powder was filled into a #3 HPMC capsule. A plastitape RSO1 low resistance dry powder inhaler was used for the testing. An about 3.1 mg dose of the spray dried powder formulation was delivered. The MMAD and GSD for the spray dried powder formulation were 2.213 μm and 1.790, respectively. The fine particle fraction, which is defined as the amount of active particles with an MMAD less than 5 μm, was 83.591%. Results from particle characterization can be seen in FIG. 1, FIG. 2, and FIG. 3.

Concentration of Compound 1: The concentration of Compound 1 in the spray dried formulation was determined by UV absorption and was found to be 46.6% w/w.

Concentration of Treprostinil: A high performance liquid chromatography (HPLC) method was developed and used to determine the concentration of Treprostinil in the spray dried powder formulation. The materials used for this method are shown below in Table 1.

TABLE 1
Materials and chemicals used in the developed HPLC method

| Product | Manufacturer | Lot number |
|---|---|---|
| Treprostinil | Tocris Bioscience | 1AV204181 |
| PK10571 | IRIX Pharmaceuticals | 15-03721-S |
| Acetonitrile | BDH, VWR Chemicals | 15K301130 |
| Water, HPLC grade | Beantown Chemicals BTC | 92000045 |
| TFA acid | Sigma-Aldrich | BCBP4740V |
| Methanol | Sigma-Aldrich | SHBF5355V |

Samples used for analysis were prepared by dissolving 10 mg of the spray dried powder formulation in 10 ml of methanol. Treprostinil standards with concentrations of 0.1, 1.0, 10, and 100 μg/ml were prepared by serial dilution using a stock solution of 1000 μg/mL in methanol. The calibrators were used to determine the linearity of the method. Details on the developed HPLC method are shown below in Table 2.

TABLE 2
Analytical HPLC method

| Instrument | | |
|---|---|---|
| Parameter | Description | |
| Column | Waters X Bridge C18, 3.5 um, 4.6 mm × 150 mm | |
| UV wavelenght | 217 | nm |
| Run time | 30 | minutes |
| Column Temperature | 20° | C. |
| Flow rate | 1000 | μg/mL |
| Injection volume | 10 | μl |

| | |
|---|---|
| Needle wash | 80% methanol/20% Water |
| Mobile phases | A: 0.1% TFA acid in Water |
| | B: 0.1% TFA acid in acetonitrile |

| | Time (min) | % A | % B |
|---|---|---|---|
| Mobile Phase gradient | 0.0 | 75 | 25 |
| | 5.0 | 75 | 25 |
| | 15.0 | 35 | 65 |
| | 25.0 | 35 | 65 |
| | 25.1 | 75 | 25 |
| | 30 | 75 | 25 |

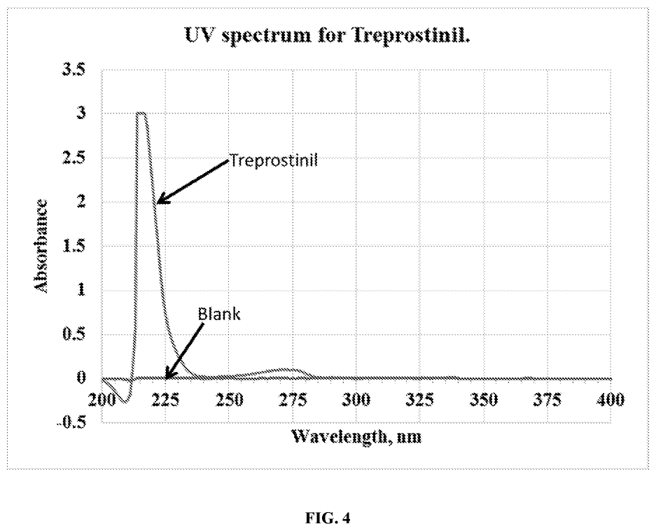

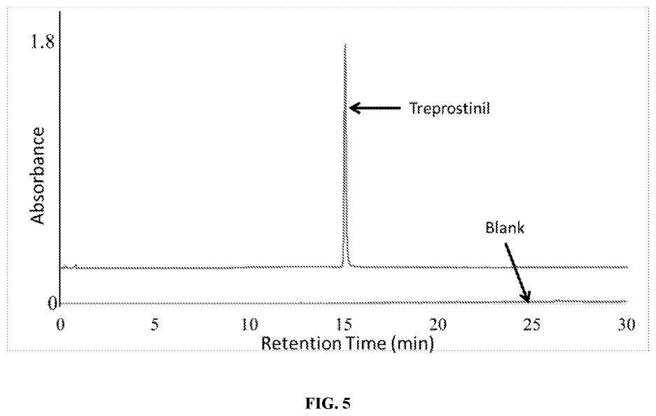

To determine the specificity of the developed HPLC method a UV experiment using 10 10 μg/ml Treprostinil was performed. The UV spectrum showed the maximum absorbance of Treprostinil at 217 nm, as shown in FIG. 4. This wavelength was used to optimize the developed HPLC method. The HPLC chromatograms showed the retention time of Treprostinil at 15.2 minutes. After running the blank and pure spray dried powder, the results showed no interference at the retention time of Treprostinil, as shown in FIG. 5.

The system suitability was determined by analyzing 10 μg/ml of Treprostinil 6 times before running the calibration curve and after the last sample. The % CV for the first six injections of 10 μg/mL was 4% for the first 6 standards and 4.8% for all of the standards, which is less than the 10% CV recommended by the United State of Pharmacopeia for HPLC experiments.

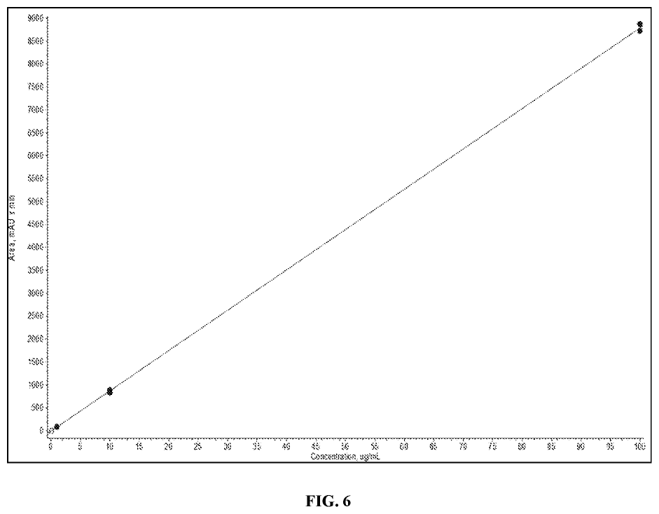

The concentration of Treprostinil in the spray dried formulation was determined using a calibration curve from 0.1 to 100 μg/ml. Each calibrator was run in triplicate. The acceptance criteria for % CV was ≤15 and the accuracy was 100±15. The standard samples met the establish specifications. The experimental lower limit of quantification (LLOQ) was 0.1 and the lower limit of detection (LLOD) was 0.05. Linear fitting of the calibration curve showed an equation of y=87.9X−7.29, r=0.9999, as shown in FIG. 6. Further information regarding the calibration curve is presented below in Table 3.

TABLE 3
Treprostinil calibration curve parameters

| Expected | | Standard | | |
|---|---|---|---|---|
| Concentration, | | Deviation, | | |
| μg/mL | Mean, μg/mL | μg/mL | % CV | Accuracy |
| 0.1 | 0.10 | 0.01 | 5.20 | 101.63 |
| 1 | 0.94 | N/A | N/A | 93.91 |
| 10 | 10.20 | 0.74 | 0.07 | 101.98 |
| 100 | 99.99 | 1.17 | 1.17 | 99.99 |

LLOQ = 0.10 μg/mL
Acceptance Criteria: % Accuracy must be 100 ± 15 with a % CV ≤ 15, except at the LLOQ where % accuracy must be 100 ± 20 and % CV ≤ 20.

The concentration of Treprostinil in the spray dried powder was determined in triplicate. The spray dried powder formulation containing Treprostinil was prepared with a final concentration of 1 mg/ml (1.230 mg of sample diluted with 1.2 mL of Methanol). This sample was then diluted 20 fold and subjected to the developed HPLC method. As can be seen below in Table 4, the average concentration of Treprostinil in the 1 mg/mL solution was found to be 421.9 μg/ml. Thus, the average concentration of Treprostinil in the spray dried formulation was 421.9 μg/mg.

TABLE 4
Calculated concentration of Treprostinil

| | | Concentration | Concentration | Average |
|---|---|---|---|---|
| | | of HPLC | of sample after | Concentration |
| Repetition | Area | sample, μg/mL | dilution factor | μg/mL |
| 1 | 1800 | 20.6 | 411.3 | 421.9, % CV = 3% |
| 2 | 1910 | 21.8 | 436.3 | |
| 3 | 1830 | 20.9 | 418.1 | |

### Example 2: Design of a Study on the Effects of a Spray Dried Formulation Containing Compound 1 and a Prostanoid in Animal Models of Vasoconstriction

A person skilled in the art would consider experiments to examine the effect of a spray dried powder containing both a PDGFR receptor kinase inhibitor such as Compound 1 and a prostanoid such as Treprostinil in animal models of pulmonary vasoconstriction. One such model consists of an intravenous infusion of the thromboxane A2 receptor agonist U46619 in the rat. The thromboxane A2 receptor agonist U46619 is infused at 1000 ng/min or at other concentrations. Either vehicle powder, Compound 1 powder, or Compound 1/Treprostinil powder is insufflated into the lungs of the rats at various doses before, during, or after the U46619 infusion. The change in right ventricular end systolic pressure is monitored to determine whether the Compound 1 and Compound 1/Treprostinil powders block the U46619 induced pulmonary vasoconstriction.

Other experiments can be done to examine the effect of a spray dried powder containing both a PDGFR kinase inhibitor such as Compound 1 and a prostanoid such as Treprostinil in animal models of PAH. One such animal model is the SU5416/hypoxia rat model. In this model, pulmonary hypertension is induced by a subcutaneous injection of SU5416 20 mg/kg followed by exposure to hypoxia at an oxygen concentration of 10% for three weeks. After removal from the hypoxia chamber, animals are dosed with the Compound 1/Treprostinil spray dried powder by passive inhalation. The effect of treatment compared to vehicle is determined by measuring right ventricular or pulmonary artery pressures at the end of the study period. The effect of treatment on pulmonary vascular remodeling is examined by histological and morphometric analyses. Other models such as the monocrotaline rat model of pulmonary hypertension are studied.

What is claimed is:

1. A pharmaceutical formulation comprising:
a. a prostanoid; and
b. a compound of the formula: 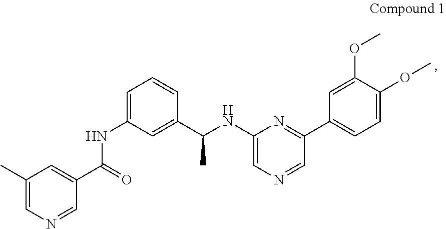
or a pharmaceutically acceptable salt thereof;
wherein the pharmaceutical formulation is a spray dried powder formulation comprising a plurality of particles with a mass median aerodynamic diameter of about 1 micron to about 5 microns.

2. The pharmaceutical formulation of claim 1, wherein the plurality of particles has a geometric standard deviation of about 1 to about 3.

3. The pharmaceutical formulation of claim 1, wherein the spray dried powder formulation has a fine particle fraction of about 70% to about 99%.

4. The pharmaceutical formulation of claim 1, further comprising a pharmaceutically-acceptable excipient.

5. The pharmaceutical formulation of claim 4, wherein the pharmaceutically-acceptable excipient is leucine or a pharmaceutically acceptable salt thereof.

6. The pharmaceutical formulation of claim 4, wherein the pharmaceutically-acceptable excipient is lactose.

7. The pharmaceutical formulation of claim 4, wherein the pharmaceutically-acceptable excipient is a phospholipid.

8. The pharmaceutical formulation of claim 1, wherein the prostanoid is present in an amount of about 5 μg to about 500 μg.

9. The pharmaceutical formulation of claim 1, wherein the prostanoid is present in an amount of about 6 μg to about 54 μg.

10. The pharmaceutical formulation of claim 1, wherein the prostanoid is present in an amount of about 25 μg to about 250 μg.

11. The pharmaceutical formulation of claim 1, wherein Compound 1 is present in an amount of about 46.6% w/w.

12. The pharmaceutical formulation of claim 1, wherein the prostanoid is present in an amount of about 408 μg/mg.

13. The pharmaceutical formulation of claim 1, wherein the prostanoid is Treprostinil.

14. The pharmaceutical formulation of claim 1, wherein the prostanoid is epoprostenol.

15. The pharmaceutical formulation of claim 1, wherein the prostanoid is iloprost.

16. The pharmaceutical formulation of claim 1, wherein the prostanoid is beraprost.

17. The pharmaceutical formulation of claim 1, wherein the prostanoid is selexipag.

18. The pharmaceutical formulation of claim 1, wherein the prostanoid is ralinepag.

19. The pharmaceutical formulation of claim 1, wherein the prostanoid is alprostadil.

20. The pharmaceutical formulation of claim 1, wherein the prostanoid is thromboxane A2.

21. The pharmaceutical formulation of claim 1, wherein the prostanoid is thromboxane B2.

22. The pharmaceutical formulation of claim 1, wherein the prostanoid is PGI₂.

23. A method of treating a pulmonary disorder, the method comprising administering to a subject in need thereof a therapeutically effective amount of a pharmaceutical formulation comprising:
a. a prostanoid; and
b. a compound of the formula: 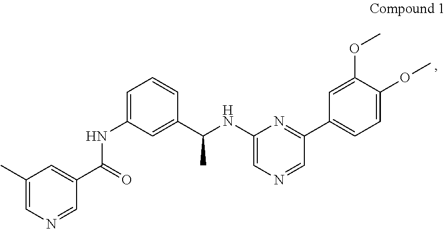
or a pharmaceutically acceptable salt thereof;
wherein the pharmaceutical formulation is a spray dried powder formulation comprising a plurality of particles with a mass median aerodynamic diameter of about 1 micron to about 5 microns.

24. The method of claim 23, wherein the administering is by a dry powder inhaler.

25. The method of claim 23, wherein the administering is by an atomizer.

26. The method of claim 23, wherein the administering is by a nebulizer.

27. The method of claim 23, wherein the administering is nasal.

28. The method of claim 23, wherein the plurality of particles has a geometric standard deviation of about 1 to about 3.

29. The method of claim 23, wherein the spray dried powder formulation has a fine particle fraction of about 70% to about 99%.

30. The method of claim 23, wherein the pharmaceutical formulation further comprises a pharmaceutically-acceptable excipient.

31. The method of claim 30, wherein the pharmaceutically-acceptable excipient is leucine or a pharmaceutically acceptable salt thereof.

32. The method of claim 30, wherein the pharmaceutically-acceptable excipient is lactose.

33. The method of claim 30, wherein the pharmaceutically-acceptable excipient is a phospholipid.

34. The method of claim 23, wherein the prostanoid is present in an amount of about 5 μg to about 500 μg.

35. The method of claim 23, wherein the prostanoid is present in an amount of about 6 μg to about 54 μg.

36. The method of claim 23, wherein the prostanoid is present in an amount of about 25 μg to about 250 μg.

37. The method of claim 23, wherein Compound 1 is present in an amount of about 46.6% w/w.

38. The method of claim 23, wherein the prostanoid is present in an amount of about 408 μg/mg.

39. The method of claim 23, wherein the prostanoid is Treprostinil.

40. The method of claim 23, wherein the prostanoid is epoprostenol.

41. The method of claim 23, wherein the prostanoid is iloprost.

42. The method of claim 23, wherein the prostanoid is beraprost.

43. The method of claim 23, wherein the prostanoid is selexipag.

44. The method of claim 23, wherein the prostanoid is ralinepag.

45. The method of claim 23, wherein the prostanoid is alprostadil.

46. The method of claim 23, wherein the prostanoid is PGI₂.

47. A pharmaceutical formulation comprising:
a. leucine or a pharmaceutically acceptable salt thereof
b. Treprostinil; and
c. a compound of the formula: 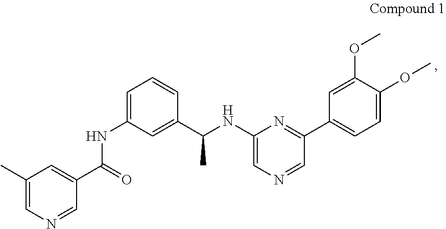
or a pharmaceutically acceptable salt thereof;
wherein:
i. the pharmaceutical formulation is a spray dried powder formulation comprising a plurality of particles with a mass median aerodynamic diameter of about 2.21 μm, a geometric standard deviation of about 1.79, and a fine particle fraction of about 83.6%,
ii. Compound 1 is present in an amount of about 46.6% w/w; and
iii. Treprostinil is present in an amount of about 408 μg/mg.

48. A method of treating a pulmonary disorder, the method comprising nasally administering to a subject in need thereof a therapeutically effective amount of a pharmaceutical formulation comprising:
a. leucine or a pharmaceutically acceptable salt thereof
b. Treprostinil; and
c. a compound of the formula: 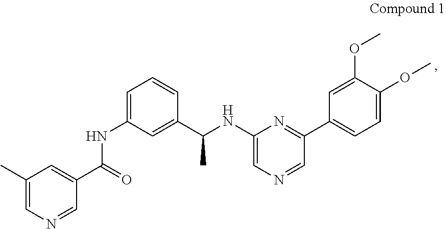
or a pharmaceutically acceptable salt thereof;
wherein:
i. the pharmaceutical formulation is a spray-dried powder formulation comprising a plurality of particles with a mass median aerodynamic diameter of about 2.21 μm, a geometric standard deviation of about 1.79, and a fine particle fraction of about 83.6%,
ii. Compound 1 is present in an amount of about 46.6% w/w; and
iii. Treprostinil is present in an amount of about 408 μg/mg.
